(19) United States
(12) Patent Application Publication
(10) Pub. No.: US 2019/0298611 A1
(43) Pub. Date: Oct. 3, 2019

(54) DEVICES AND SYSTEMS FOR CONTAMINANT-FREE ENGAGEMENT AND FLUID TRANSFER OF PHARMACEUTICAL VESSELS AND PHARMACEUTICAL ADMINISTRATION DEVICES

(71) Applicant: Mikael Naygauz, Forrest Hills, NY (US)

(72) Inventor: Mikael Naygauz, Forrest Hills, NY (US)

(21) Appl. No.: 16/254,576

(22) Filed: Jan. 22, 2019

Related U.S. Application Data
(60) Provisional application No. 62/649,483, filed on Mar. 28, 2018.
(60) Provisional application No. 62/735,023, filed on Sep. 22, 2018.
(60) Provisional application No. 62/794,744, filed on Jan. 21, 2019.

(51) Int. Cl. A61J 1/20; A61J 1/10; A61J 1/14; A61M 5/178; A61L 2/18

(52) U.S. Cl. CPC A61J 1/2048; A61J 1/10; A61J 1/1406; A61L 2202/23; A61J 1/2089; A61M 5/1782; A61L 2/18; A61J 1/2096

(57) ABSTRACT

The present invention relates, in some embodiments thereof, to systems, devices and methods allowing for an engagement of two or more vessels or devices in a decontaminated manner. In some embodiments of the invention, the systems and devices of the invention include a first connection interface attached to a first vessel and a second connection interface attached to a second vessel, wherein the first connection interface and the second connection interface are configured to allow for an engagement between the first vessel and the second vessel, and wherein the first and second connection interfaces are further configured to externally displace from the engagement between the first vessel and the second vessel while a hermetically sealed connection is maintained between the first vessel and the second vessel.

### CROSS REFERENCE TO RELATED APPLICATIONS

This application claims the benefit of U.S. Provisional Patent Application Nos. 62/649,483 filed Mar. 28, 2018; 62/735,023 filed Sep. 22, 2018; and 62/794,744 filed Jan. 21, 2019. The contents of the above applications are all incorporated by reference as if fully set forth herein in their entirety.

### FIELD OF THE INVENTION

The present invention relates, in some embodiments thereof, to systems, devices, containers, and methods allowing for an engagement of pharmaceutical vessels or pharmaceutical administration devices in a decontaminated manner.

### BACKGROUND OF THE INVENTION

The medicinal practice routinely involves administration of medical substances, such as, medicaments, fluids, nutritional substances and the alike, to patients or animals. The preparation and/or administration of such medicinal substances typically involves one or more transfers of those substances between pharmaceutical vessels or administration devices (such as, vials, syringes, infusion lines, connectors, etc.). Each such act of transferring substances between vessels or devices exposes the connection interfaces of the vessels and accordingly the medical substances themselves to contaminants present in ambient air or ambient air particles (e.g., bacteria, viruses, funguses, spores, pyrogens, dirt). In addition, connection interfaces are further prone to contaminations due to physical contact of the interfaces, for example, with nonsterile gloves, or devices.

Such contaminations are a major problem in the healthcare setting since contaminants, once invading within medicinal substances, may pose substantial danger if administered intracorporeally to patients.

Typical connection interfaces of pharmaceutical vessels or pharmaceutical administration devices include rubber bungs and/or stoppers covered by a cap and/or seal that can be flicked off and/or are removed prior to usage thereof. These rubber bungs/stoppers are used to allow penetration by a needle attached to a syringe or by other medical connectors. When the cap and/or seal is flicked off and/or removed, the rubber bung and/or stopper is exposed to ambient air and to contaminants present therein. Accordingly, exposure of connection interfaces to ambient air may involve contamination of the interfaces with dust, dirt, bacteria, fungus, viruses, and consequently contamination of a beneficial substance to be provided to a patient.

Existing systems include U.S. Pat. No. D720,067; U.S. Pat. No. D717,947; U.S. Pat. No. D703,812; U.S. Pat. No. D690,418; U.S. Pat. No. D639,939; U.S. Pat. No. D637,713; U.S. Pat. No. 9,790,011; U.S. Pat. No. 9,775,777; U.S. Pat. No. 9,561,326; U.S. Pat. No. 9,493,281; U.S. Pat. No. 9,492,353; U.S. Pat. No. 9,309,020; U.S. Pat. No. 9,173,816; U.S. Pat. No. 9,168,203; U.S. Pat. No. 9,162,803; U.S. Pat. No. 9,039,672; U.S. Pat. No. 8,926,583; U.S. Pat. No. 8,827,978; U.S. Pat. No. 8,790,330; U.S. Pat. No. 8,662,985; U.S. Pat. No. 8,657,803; U.S. Pat. No. 8,622,985; U.S. Pat. No. 8,562,583; U.S. Pat. No. 8,545,475; U.S. Pat. No. 8,523,838; U.S. Pat. No. 8,491,563; U.S. Pat. No. 8,480,646; U.S. Pat. No. 8,449,521; U.S. Pat. No. 8,381,776; U.S. Pat. No. 8,336,587; U.S. Pat. No. 8,328,772; U.S. Pat. No. 8,287,513; U.S. Pat. No. 8,225,826; U.S. Pat. No. 8,075,550; U.S. Pat. No. 8,029,747; U.S. Pat. No. 7,998,134; U.S. Pat. No. 7,975,733; U.S. Pat. No. 7,942,860; U.S. Pat. No. 7,867,215; U.S. Pat. No. 7,744,581; U.S. Pat. No. 7,731,678; U.S. Pat. No. 7,387,216; U.S. Pat. No. 7,306,584; U.S. Pat. No. 6,875,203; U.S. Pat. No. 6,729,370; U.S. Pat. No. 6,715,520; U.S. Pat. No. 6,602,239; U.S. Pat. No. 6,409,708; U.S. Pat. No. 6,343,629; U.S. Pat. No. 6,162,199; U.S. Pat. No. 6,113,583; U.S. Pat. No. 6,063,068; U.S. Pat. No. 5,893,397; U.S. Pat. No. 5,876,380; U.S. Pat. No. 5,832,971; U.S. Pat. No. 5,807,374; U.S. Pat. No. 5,746,733; U.S. Pat. No. 5,569,235; U.S. Pat. No. 5,462,535; U.S. Pat. No. 5,405,326; U.S. Pat. No. 5,292,318; U.S. Pat. No. 5,279,582; U.S. Pat. No. 4,944,723; U.S. Pat. No. 4,932,947; U.S. Pat. No. 4,932,937; U.S. Pat. No. 4,919,657; U.S. Pat. No. 4,915,701; U.S. Pat. No. 4,826,489; U.S. Pat. No. 4,673,404; U.S. Pat. No. 4,564,054; U.S. Pat. No. 3,610,241; U.S. Pat. No. 3,605,743; U.S. Pat. No. 3,587,575; U.S. Pat. No. 3,583,399; U.S. Pat. No. 3,578,037; U.S. Pat. No. 3,556,099; U.S. Pat. No. 3,552,387; U.S. Pat. No. 3,406,686; U.S. Pat. No. 3,380,450; U.S. Pat. No. 3,375,825; U.S. Pat. No. 3,342,180; U.S. Pat. No. 3,330,282; U.S. Pat. No. 3,330,281; U.S. Pat. No. 3,306,290; U.S. Pat. No. 3,255,752; U.S. Pat. No. 3,253,592; U.S. Pat. No. 3,076,456; U.S. Pat. No. 2,972,991; U.S. Pat. No. 2,922,419; US 20160262982; US 20160038373; US 20150209568; US 20140183196; US 20140016570; US 20140007973; US 20140000754; US 20130184672; US 20130006200; US 20120209238; US 20120209218; US 20120203194; US 20110284561; US 20110186177; US 20110125128; US 20110108158; US 20110098647; US 20100249745; US 20100198182; US 20100152669; US 20100147402; US 20100036319; US 20100004602; US 20090057258; US 20080312634; US 20080223484; US 20080171981; US 20060276759; US 20050215976; US 20030199847; US 20030187420; US 20020130100; US 20020115981; US 20020099354; ES2577377T3; EP2852367B1; EP2666513; EP2155141B1.

In order to overcome this obstacle, the current medical practice involves swabbing the surface of a connection interface with a disinfecting agent, such as 70% isopropyl alcohol, prior to accessing the connection interface. Other methods include i.v. (intravenous) rooms which are used for the sterile preparation of i.v. medications. Such rooms, to keep medicinal preparations as sterile as possible, are equipped with special instruments including, hoods with air filtration systems (e.g., HEPA filters), ventilation systems and air pressure systems. Additionally, those rooms necessitate that the medical staff working in these rooms are properly garmented, are properly trained, and require aseptic techniques, and employ quality control and validation processes. These systems require regular upkeep by certified personnel and require regular cleaning. These systems are not foolproof and periodically become contaminated with bacteria, fungus, and/or other contaminants. These systems are therefore expensive, labor intensive, and require regular maintenance and testing to assure that they are operating effectively. The above described systems and methods are either cumbersome and expensive or inefficient in addressing the problem of reducing/eliminating contaminants on connection interfaces.

Thus, there is a long felt and unmet need for pharmaceutical vessels, devices, systems and/or methods that afford transfer of medical substances in a sterile manner. There is a need for reliable, user friendly and cost-effective solutions allowing contaminant-free engagement of vessels and devices for drug preparation and administration processes.

### SUMMARY OF THE INVENTION

Objects of the invention are achieved by providing systems, devices, containers, vessels and methods for engaging medical devices, such as vessels, in a decontaminated manner.

Objects of the invention are achieved by providing systems, devices, vessels and methods which are directed to transferring medical substances in an efficient, user-friendly and sterile manner.

The present invention is based on two or more connection interfaces, each of which is attached to a vessel. The connection interfaces are configured to connect between two or more medical vessels and while doing so, isolate and entrap therebetween ambient air particles (and contaminants present in ambient air). Such devices, vessels, and systems allow for a hermetic and sterile connection between the vessels.

In one or more embodiments, at least a portion of the first and/or second connection interface is externally exposed, and the first vessel and second vessel are configured to engage with each other via the exposed portions of the connection interfaces, wherein once attached to each other, the exposed portions displace internally within a fluid free compartment in the first and/or second vessels.

In one or more embodiments, at least a portion of the first and/or second connection interface is externally exposed, and the first vessel and second vessel are configured to engage with each other via the exposed portions of the connection interfaces, wherein once attached to each other, the exposed portions displace externally to the first and/or second vessels.

In one or more embodiments, the connection interface includes an externally exposed portion and a portion concealed within the vessel, and the first vessel and second vessel are configured to engage with each other via the exposed portions of the connection interfaces, wherein once attached to each other, the exposed portions displace externally and are replaced by the internal concealed portions.

In a first aspect, the present invention provides a decontamination system for establishing a fluid passageway between vessels, the system comprising:

a first connection interface attached to and/or sealing an aperture of a first vessel; and

a second connection interface attached to and/or sealing an aperture of a second vessel,

wherein at least a portion of the first and/or second connection interface is externally exposed, wherein the first vessel and the second vessel are configured to engage with each other via the exposed portions of the connection interfaces, and wherein the exposed portions of the connection interfaces are further configured to displace from the engagement between the first vessel and the second vessel while a hermetically sealed connection is maintained between the first vessel and the second vessel.

In another aspect, the present invention provides a decontamination system for establishing a fluid passageway between vessels, the system comprising:

a first connection interface attached to a first vessel; and

a second connection interface attached to a second vessel,

wherein at least a portion of the first and/or second connection interface is externally disposed on the first and/or second vessel, wherein the first vessel and the second vessel are configured to engage with each other via the external portion of the connection interfaces, and wherein the external portion of the connection interfaces are further configure to displace from the engagement between the first vessel and the second vessel while a hermetically sealed connection is maintained between the first vessel and the second vessel.

In another aspect, the present invention provides a system for the engagement of two vessels, the system comprising:

a first vessel having a first connection interface, the first connection interface characterized in that a portion thereof is concealed within the first vessel and another portion thereof is exposed to ambient air; and

a second vessel having a second connection interface, the second connection interface characterized in that a portion thereof is concealed within the second vessel and another portion thereof is exposed to ambient air,

wherein the first vessel and second vessel are configured to engage with each other via the exposed portions of the connection interfaces, wherein once attached to each other, the exposed portions displace externally or internally within the respective vessel and are replaced by the internal concealed portions.

In one or more embodiments, a portion of the connection interface is disposed within the vessel and a portion of the connection interface is exposed to ambient air.

In one or more embodiments, at least one of the first connection interface and the second connection interface is a film, a plastic, or a metal alloy.

In one or more embodiments, at least the exposed portion of the connection interface displaces externally. In one or more embodiments, at least the exposed portion of the connection interface displaces internally within a vessel.

In one or more embodiments, the connection interface further comprises an internal portion concealed within the vessel. In one or more embodiments, the internal portion is a first end, a second end or both ends of the connection interface.

In one or more embodiments, following the engagement between the vessels, at least the exposed portions of the interfaces are displaced from the engagement and replaced by the concealed portions, allowing a sterile connection between the vessels.

In one or more embodiments, at least one of the first and second vessels include(s) a fluid compartment and a fluid-free compartment. In one or more embodiments, at least one of the first and second vessels is a bag. In one or more embodiments, at least one of the first and second vessels is a bottle. In one or more embodiments, the first and second vessels are containers. In one or more embodiments, the first vessel is a syringe and the second vessel is a container.

In one or more embodiments, the connection interface further comprises an internal portion concealed within the fluid-free compartment. In one or more embodiments, the internal portion is a first end, a second end or both ends of the connection interface.

In one or more embodiments, following the engagement between the vessels, at least the exposed portions of at least one of the connection interfaces displaces internally within the fluid-free compartment.

In one or more embodiments, at least one of the first and second vessels include(s) a rotating mechanism, wherein the connection interface connected to the rotating mechanism and the rotating mechanism configured to displace the connection interface. In one or more embodiments, at least one of the first and second vessels include(s) a rotating mechanism configured to rotate within the vessel. In one or more embodiments, at least one of the first and second vessels include(s) a rotating mechanism configured to rotate within the vessel, wherein the rotating mechanism includes a conduit, the conduit allows the fluid passageway upon the rotation. In one or more embodiments, the conduit includes a valve for controlling the fluid passageway between the vessels. In one or more embodiments, the rotating mechanism includes a handle extending externally from an opening of the vessel and configured to be rotated by a user such to allow the displacement of the first connection interface and/or second connection interface. In one or more embodiments, the rotating mechanism includes a handle extending externally from an opening of the vessel and configured to be rotated by a user such to allow the displacement of the first connection interface and/or second connection interface.

In one or more embodiments, the first and/or second vessel includes a port surrounding the aperture, wherein the engagement between the vessels is via the port.

In one or more embodiments, the system further comprises a pressing member on the first vessel and/or the second vessel configured to press the first vessel and/or second vessel, against a wall of the vessel where the connection interface resides, thereby allowing a hermetically sealed aperture. In one or more embodiments, the vessel has a pressing member configured to press the connection interface against an interior, and/or optionally an exterior, wall of the vessel. In one or more embodiments, the pressing member may be made of a rubber material and/or an elastomeric material.

In one or more embodiments, a hermetically sealed connection is maintained between the first vessel and the second vessel following the vessels' engagement.

In one or more embodiments, upon the engagement between the first vessel and the second vessel, the exposed portions of the first connection interface and the second connection interface are configured to displace with respect to the first vessel and the second vessel, thereby removing contaminants between the vessels. In one or more embodiments, the decontamination system results in air particle-free engagement of the first vessel and the second vessel, wherein air particles interfacing (exposed to) the interfaces are entrapped by the exposed portions of the interfaces.

In one or more embodiments, the system further comprising an engagement mechanism configured to provide a secure engagement between the vessels. In one or more embodiments, the engagement mechanism is located on the first vessel and/or on the second vessel. In one or more embodiments, the engagement mechanism is selected from a thread, a luer, an adhesive, a clamping arm/rail, a clasping member, a retention member, a hook and a dent, a ratchet teeth mechanism and a combination thereof. In one or more embodiments, upon the vessels' engagement via the engagement mechanism, an airtight seal is formed between the vessels. In one or more embodiments, when the first vessel and the second vessel engage via the engagement mechanism, a sealed aperture is formed between the vessels. In one or more embodiments, the sealed aperture is configured to displace at least one connection interface through the sealed aperture while maintaining the hermetically sealed connection between the first vessel and the second vessel. In one or more embodiments, the engagement mechanism is a thread on the first vessel configured to engage a complementary thread on the second vessel. In one or more embodiments, the thread of at least one of the threads and complementary thread is a luer. In one or more embodiments, an aperture is disposed between at least one of the threads of the first and/or the second vessel, wherein the aperture is configured to allow displacement of at least one connection interface of the first and/or the second vessel through the aperture. In one or more embodiments, at least one of the first and second connection interfaces displaces between ratchet teeth of the first vessel and/or the second vessel.

In one or more embodiments, the system further comprising a rail mechanism on at least one of the first and second vessels, the rail mechanism configured to provide the displacement of the connection interface. In one or more embodiments, the first connection interface and/or the second connection interface are externally displaced via a sliding motion. In one or more embodiments, the first connection interface and the second connection interface displace at, or about the same time.

In one or more embodiments, the system further comprising a tab on at least one of the first connection interface and the second connection interface, the tab configured to allow for an external displacement of the first connection interface and the second connection interface.

In one or more embodiments, the tab is configured to be pulled axially by a user, the axial pulling of the tab configured to displace the first connection interface and the second connection interface from the first vessel and the second vessel while maintaining the hermetically sealed connection between the first vessel and the second vessel.

In one or more embodiments, the system further comprising a piercing member on the first vessel and/or the second vessel configured to pierce a sealed aperture of the first vessel and/or the second vessel, thereby allowing fluid passageway between the vessels. In one or more embodiments, the piercing member is a needle. In one or more embodiments, the needle is a hollow needle.

In one or more embodiments, an audible sound is emitted when the first vessel engages with the second vessel and/or when the first interface and the second interface are displaced.

In one or more embodiments, at least one of the first and second connection interfaces is configured to be pulled by a user. In one or more embodiments, the pulling of one of the connection interfaces facilitates the movement of the other connection interface.

In another aspect, the present invention provides a system for the engagement of two vessels, the system comprising:

a first vessel having a first connection interface, the first connection interface characterized in that a portion thereof is concealed within the first vessel and another portion thereof is exposed to ambient air; and

a second vessel having a second connection interface, the second connection interface characterized in that a portion thereof is concealed within the second vessel and another portion thereof is exposed to ambient air,

wherein the first vessel and second vessel are configured to engage with each other via the exposed portions of the connection interfaces, wherein once attached to each other, the exposed portions displace externally and are replaced by the internal concealed portions.

In another aspect, the present invention provides a system for the engagement of two vessels, the system comprising:

a first vessel having a first connection interface, the first connection interface characterized in that a portion thereof is concealed within the first vessel and another portion thereof is exposed to ambient air; and

a second vessel having a second connection interface, the second connection interface characterized in that a portion thereof is concealed within the second vessel and another portion thereof is exposed to ambient air,

wherein the first vessel and second vessel are configured to engage with each other, wherein once attached to each other, the exposed portions displace internally within the first or second vessel and are replaced by the internal concealed portions.

In another aspect, the present invention provides a system for the engagement of two vessels, the system comprising:

a first vessel having a first connection interface, the first connection interface characterized in that a portion thereof is concealed within the first vessel and another portion thereof is exposed to ambient air; and

a second vessel having a second connection interface, the second connection interface characterized in that a portion thereof is concealed within the second vessel and another portion thereof is exposed to ambient air,

wherein the first vessel and second vessel are configured to engage with each other, wherein once attached to each other, the exposed portions displace internally within the first or second vessel.

In another aspect, the present invention provides a system for the engagement of two vessels, the system comprising:

a first vessel having a first connection interface, the first connection interface characterized in that a portion thereof is concealed within the first vessel and another portion thereof is exposed to ambient air; and

a second vessel having a second connection interface, the second connection interface characterized in that a portion thereof is concealed within the second vessel and another portion thereof is exposed to ambient air,

wherein the first vessel and second vessel are configured to engage with each other, wherein once attached to each other, the exposed portions displace internally within the respective first and second vessel and are replaced by the internal concealed portions.

In another aspect, the present invention provides a system for the engagement of two vessels, the system comprising:

a first vessel having a first connection interface, the first connection interface characterized in that a portion thereof is concealed within the first vessel and another portion thereof is exposed to ambient air; and

a second vessel having a second connection interface, the second connection interface characterized in that a portion thereof is concealed within the second vessel and another portion thereof is exposed to ambient air,

wherein the first vessel and second vessel are configured to engage with each other, wherein once attached to each other, the exposed portions displace internally within the respective first and second vessel.

In a further aspect, the present invention provides a vessel having a connection interface, the connection interface characterized in that a portion of the connection interface is concealed within the vessel and a portion of the connection interface is exposed to ambient air.

In one or more embodiments, the concealed portion of the connection interface is sterile.

In one or more embodiments, the concealed portion of the connection interface is disposed within a port of the vessel.

In one or more embodiments, the concealed portion of the connection interface is disposed within the vessel body.

In one or more embodiments, the vessel is a container.

In one or more embodiments, the vessel is a bag.

In one or more embodiments, the vessel is a bottle.

In one or more embodiments, the vessel is a vial.

In one or more embodiments, the vessel is a syringe.

In one or more embodiments, the vessel is a connector.

In a further aspect, the present invention provides a vessel having a connection interface, the connection interface characterized in that a portion of the connection interface is concealed within the vessel and a portion of the connection interface is exposed to ambient air, the connection interface further configured to be displaced externally from the vessel.

In a further aspect, the present invention provides a vessel having a connection interface, the connection interface characterized in that a portion of the connection interface is concealed within the vessel and a portion of the connection interface is exposed to ambient air, the exposed to ambient air portion of the connection interface further configured to be displaced externally from the vessel and replaced with the concealed portion of the connection interface.

In one or more embodiments, the concealed portion of the connection interface is sterile.

In one or more embodiments, the concealed portion of the connection interface is disposed within a port of the vessel.

In one or more embodiments, the concealed portion of the connection interface is disposed within the vessel body.

In one or more embodiments, the vessel is a container.

In one or more embodiments, the vessel is a bag.

In one or more embodiments, the vessel is a bottle.

In one or more embodiments, the vessel is a vial.

In one or more embodiments, the vessel is a syringe.

In one or more embodiments, the vessel is a connector.

In a further aspect, the present invention provides a vessel having a connection interface, the connection interface characterized in that a portion of the connection interface is concealed within the vessel and a portion of the connection interface is exposed to ambient air, the connection interface further configured to be displaced internally into a vessel.

In one or more embodiments, the concealed portion of the connection interface is sterile.

In one or more embodiments, the concealed portion of the connection interface is disposed within a port of the vessel.

In one or more embodiments, the concealed portion of the connection interface is disposed within the vessel body.

In one or more embodiments, the vessel is a container.

In one or more embodiments, the vessel is a bag.

In one or more embodiments, the vessel is a bottle.

In one or more embodiments, the vessel is a vial.

In one or more embodiments, the vessel is a syringe.

In one or more embodiments, the vessel is a connector.

In a further aspect, the present invention provides a vessel having a connection interface, the connection interface characterized in that a portion of the connection interface is concealed within the vessel and a portion of the connection interface is exposed to ambient air, the connection interface further configured to be displaced internally into the vessel.

In one or more embodiments, the concealed portion of the connection interface is sterile.

In one or more embodiments, the concealed portion of the connection interface is disposed within a port of the vessel.

In one or more embodiments, the concealed portion of the connection interface is disposed within the vessel body.

In one or more embodiments, the vessel is a container.

In one or more embodiments, the vessel is a bag.

In one or more embodiments, the vessel is a bottle.

In one or more embodiments, the vessel is a vial.

In one or more embodiments, the vessel is a syringe.

In one or more embodiments, the vessel is a connector.

In a further aspect, the present invention provides a method of decontaminating a connection between two vessels, the method comprising the steps of:

providing a first connection interface attached to a first vessel, wherein at least a portion of the first connection interface is externally exposed;

providing a second connection interface attached to a second vessel, wherein at least a portion of the second connection interface is externally exposed,

engaging the first vessel to the second vessel; and

displacing the first connection interface and the second connection interface from the engagement between the first vessel and the second vessel.

In one or more embodiments, the step of displacing the first and/or second connection interface includes an external displacement. In one or more embodiments, at least one of the first and second connection interfaces is displaced external to a port of the first and/or second vessel.

In one or more embodiments, the step of displacing the first and/or second connection interface includes an internal displacement. In one or more embodiments, at least one of the first and second connection interfaces is displaced internally into at least one of the first and second vessel(s).

In one or more embodiments, the step of displacing the first connection interface and/or the second connection interface occurs via a rotation motion.

In one or more embodiments, the rotation motion is a horizontal rotation or a vertical rotation.

In one or more embodiments, the step of displacing the first connection interface and/or the second connection interface occurs via a sliding motion.

In one or more embodiments, the step of externally displacing the first connection interface and the second connection interface occurs via a peeling motion. In one or more embodiments, at least one of the first and second connection interfaces is attached to the first and/or second vessel via a frangible seal, wherein the frangible seal is configured to break and release the connection interface upon pulling by a user's hand.

In one or more embodiments, the step of displacing the first connection interface and the second connection interface allows to remove contaminants located between the first connection interface and the second connection interface. In one or more embodiments, the step of displacing the first connection interface and the second connection interface allows to remove contaminants located between the first vessel and the second vessel. In one or more embodiments, the first vessel and the second vessel are containers. In one or more embodiments, the first vessel is a container and the second vessel is a syringe.

In one or more embodiments, an airtight seal is formed between the first connection interface and the second connection interface prior to the displacement of the first connection interface and the second connection interface.

In one or more embodiments, when the airtight seal between the first connection interface and the second connection interface is displaced, the connection between the first vessel and the second vessel is decontaminated.

In one or more embodiments, the airtight seal between the first connection interface and the second connection interface is maintained following the displacement, allowing a hermetically sealed connection between the first vessel and the second vessel.

In one or more embodiments, displacement of the first connection interface and the second connection interface is via a rail mechanism, such that the airtight seal between the first connection interface and the second connection interface slides off the rail mechanism. In one or more embodiments, the rail mechanism is located on at least one of the first and second vessels. In one or more embodiments, at least one of the first and second connection interfaces is disposed on the rail mechanism. In one or more embodiments, at least one of the first and second connection interfaces is disposed between rails of the rail mechanism.

In one or more embodiments, the displacement of the first connection interface and the second connection interface is via a rotation mechanism, such that the airtight seal between the first connection interface and the second connection interface rotates via the rotation mechanism.

In one or more embodiments, the step of displacing the first connection interface and the second connection interface occurs at about the same time. In one or more embodiments, the step of displacing the first connection interface and the second connection interface occurs at the same time. In one or more embodiments, displacing the first connection interface facilitates the displacement of the second connection interface. In one or more embodiments, displacing the second connection interface facilitates displacment of the first connection interface.

In one or more embodiments, one or both ends of the first and/or second connection interface is internally concealed within the vessel, optionally within a port of the vessel, and wherein the step of displacement includes replacing the exposed portion(s) with the internal concealed portions, allowing for a sterile engagement between the vessels. In one or more embodiments, the internal concealed portions/ends of the connection interface(s) is sterile.

In one or more embodiments, the method further comprising connecting the first connection interface to the second connection interface via an engagement mechanism.

In one or more embodiments, the connecting of the first connection interface to the second connection interface occurs prior to displacing the first connection interface and the second connection interface from the engagement between the first vessel and the second vessel.

In one or more embodiments, the connecting of the first connection interface to the second connection interface is selected from a twisting of a thread onto a complementary thread, attaching ratchet teeth to a complementary retention member, and adhering a first connection interface to the second connection interface via an adhesive, and combinations thereof.

In one or more embodiments, the engaging of the first connection interface to the second connection interface is mediated via an adhesive disposed on at least one of the first and second connection interface.

In one or more embodiments, the adhesive is a glue and/or a tape-like sub stance.

In one or more embodiments, the engagement step is followed by a locking of the first vessel to the second vessel via push, twist, slide activation, or combinations thereof.

In one or more embodiments, the displacement step is preceded by a locking of the first interface to the second interface via push, twist, slide activation, or combinations thereof.

In one or more embodiments, connecting of the first connection interface to the second connection interface actuates a piercing member/needle to pierce the vessel surfaces.

In one or more embodiments, the method further comprising actuating a piercing member/needle to pierce the vessel surfaces.

In one or more embodiments, the method further comprising actuating a piercing member/needle to pierce the remained engaged portion of the connection interfaces.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D are schematic cut view illustrations presenting an exemplary decontamination system which comprises a first connection interface and a second connection interface, each having a sterile internal concealed portion and an externally exposed portion, the engagement between the vessels is via the exposed portions of the connection interfaces, the exposed portions configured to displace externally and are replaced by the internal concealed portions, such to allow a hermetic and decontaminated communication between the vessels, according to some embodiments of the invention.

FIG. 2 is a cut view illustration of a dual compartment device having a fluid free compartment and a fluid compartment, the device includes a rotating mechanism configured to mediate displacement of a connection interface and/or fluid passageway of the fluid in the fluid compartment, according to some embodiments of the invention.

FIGS. 3A-3B are a cut view illustration of the vessel of FIG. 2 which further includes a connection interface, the connection interface includes sterile internally concealed portions at opposing ends thereof and a middle exposed portion, according to some embodiments of the invention.

FIG. 4 is a cut view illustration of an exemplary position of the connection interface of FIG. 2, the connection interface includes a sterile internally concealed portion and an externally exposed portion, according to some embodiments of the invention.

FIGS. 5A-5D are cut view illustrations of a system that includes two vessels, each vessel has two compartments and a rotating mechanism, the vessels further include a connection interface with a sterile internally concealed portion and an externally exposed portion, according to some embodiments of the invention.

FIGS. 6A-6E are cut view illustrations of yet a further exemplary system, the system includes two vessels, each having a connection interface with an externally exposed portion, the connection interfaces configured to displace internally into the first vessel and thereby allow a fluid passageway and decontaminated engagement between the vessels, according to some embodiments of the invention.

FIG. 7 is a side view illustration of the system of FIGS. 6A-6E, the system includes a handle to maneuver rotation of the rotating mechanism, according to some embodiments of the invention.

FIGS. 8A-8B are cut view illustrations of yet a further exemplary system, the system includes an engagement mechanism in the form of adhesives, according to some embodiments of the invention.

FIG. 9 is a cut view illustration of the system of FIGS. 8A-8B which further includes a press mechanism, according to some embodiments of the invention.

FIGS. 10A-10B are cut view illustrations of yet a further exemplary system, the system includes an engagement mechanism in the form of a thread, according to some embodiments of the invention.

FIGS. 11A-11B are cut view illustrations of yet a further exemplary system, the system includes an engagement mechanism in the form of clamping arms, according to some embodiments of the invention.

FIGS. 12A-12B are cut view illustrations of yet a further exemplary system, the system includes an engagement mechanism in the form of square hooks, according to some embodiments of the invention.

FIGS. 13A-13D are cut view illustrations of yet a further exemplary system, the system includes two vessels, each having a rotating mechanism configured to rotate in a counter clock wise direction, according to some embodiments of the invention.

FIGS. 14A-14B are cut view illustrations of yet a further exemplary system, the system has two vessels, the first vessel includes a rotating mechanism configured to rotate in a counter clock wise direction, the second vessel includes a rotating mechanism configured to rotate in a clock wise direction, according to some embodiments of the invention.

FIGS. 15A-15E are cut view illustrations of yet a further exemplary system, the system has a rotating mechanism with a dent for allowing a user to actuate the rotating mechanism, according to some embodiments of the invention.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary medical devices for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention relates to devices, methods and systems allowing an engagement of medical containers or devices in a sterile manner. In one embodiment, the present invention provides connection interfaces, that may be coupled to, or integrally formed with, vessels or containers, such as bags, bottles, connectors, vials, syringes, etc. The invention provides a solution to an unmet and long felt need in the medical setting and allows connecting in a sterile manner, two or more vessels. The herein disclosed devices and systems are user friendly, cost effective and abolish the need for complicated and expensive known methods for transferring medical substances in a sterile manner. In an embodiment of the invention, the herein disclosed devices and systems are disposable. In an embodiment of the invention, the herein disclosed devices and systems are non-disposable. In an embodiment of the invention, the herein disclosed vessels and devices are intended for one-time use. In an embodiment of the invention, the herein disclosed vessels and devices are intended to be reused.

In an aspect of the invention, the devices and systems include a first connection interface attached to a first vessel and a second connection interface attached to a second vessel. The first and/or second connection interfaces may be attached to a port/aperture of the first and/or second vessel. The first and second connection interfaces are configured to allow an engagement therebetween the connection interfaces. When the connection interfaces engage each other, ambient air particles present at the vicinity thereof are entrapped within/between the engagement formed by the connection interfaces. The engagement between the connection interfaces further establishes an airtight engagement of the vessels. The connection interfaces are further configured to displace from the vessels, thereby allowing fluid flow between the vessels and contaminants-free connection of the vessels. Optionally, the connection interfaces are displaced internally within a compartment of the vessel. Optionally, the connection interfaces include an internal sterile portion concealed within the vessel and an external portion exposed to (contaminated) ambient air, wherein following a vessels' engagement the exposed portions displace externally and replaced by the internal concealed and sterile portions.

Accordingly, the herein disclosed invention allows transferring medical substances in a contaminants-free, or in a substantially contaminants-free manner. In one or more embodiments, the herein disclosed invention allows connecting vessels in a contaminants-free, or in a substantially contaminants-free manner.

In one or more embodiments, the herein disclosed invention provides a fluidic passageway or communication between medical containers in a contaminants-free, or in a substantially contaminants-free manner.

In one or more embodiments, the herein disclosed invention affords to isolate and/or entrap ambient air particles present between two, or more, vessels. In one or more embodiments, the herein disclosed invention, allows to substantially decrease the chances to introduce contamination within a medical substance, when preparing or administering medical substances.

As used herein, the term “substances” refers to various types of materials that should be kept sterile. The substances may be liquid, solid, semi-solid, or gas. In one or more embodiments, the substances are “medical substances”. As used herein the term “medical substances” refers to and encompasses any of the various medical drugs, fluids, nutritional products, solid powders, suspensions, liquids solutions and the like.

As used herein, the term “contaminant-free” is interchangeable with the term “sterile”, “disinfected”, and “decontaminated”. The term refers to substances that are free or substantially free of ambient air particles and/or pathogens, including but not limited to dirt, dust, bacteria, viruses, funguses, spores, and/or pyrogens. Typically, when less or no air is introduced within medical substances, the chances of contamination by pathogens, such as, bacteria, viruses, funguses, spores, pyrogens or the alike is completely abolished or significantly reduced.

As used herein, “substantially contaminants-free” means significantly less ambient air present when transferring medical substances with the herein disclosed vessels and systems, as compared to comparable conditions for transferring medical substances without the herein disclosed vessels and systems.

As used herein, the term “ambient air particles” is interchangeable with the term “environmental air particles” and refers to air particles present in a non-filtered or filtered environment. For example, air can be purified by filters, such as a High Efficiency Particulate Air (HEPA) filter.

As used herein, the term “connection interface” encompasses any surface, layer plane or the alike that can be attached to a vessel. The term may encompass a structure that can be coupled to a vessel and that can engage with a complementary connection interface. In an embodiment of the invention, the connection interface may seal or cover an aperture of a vessel. In an embodiment of the invention, the connection interface may seal or cover an aperture for fluid passageway of a vessel. In an embodiment of the invention, the connection interface may be a surface of a container. In an embodiment of the invention, the connection interface may be a surface of a device. In an embodiment of the invention, the connection interface may be a surface of a vessel. In an embodiment of the invention, the connection interface abuts a container. In an embodiment of the invention, the connection interface is surface mounted to a container. In an embodiment of the invention, the connection interface is surface mounted to a wall of the container. In an embodiment of the invention, the connection interface is flush mounted to a container. In an embodiment of the invention the connection interface is a vessel surface where a fluidic communication is established between the vessel and a second vessel or device.

In an embodiment of the invention, the connection interface abuts a device. In an embodiment of the invention, the connection interface is surface mounted to a device. In an embodiment of the invention, the connection interface is surface mounted to a wall of a device. In an embodiment of the invention, the connection interface is flush mounted to a device.

In an embodiment of the invention, the connection interface abuts a vessel. In an embodiment of the invention, the connection interface is surface mounted to a vessel. In an embodiment of the invention, the connection interface is surface mounted to a wall of a vessel. In an embodiment of the invention the connection interface is flush mounted to a vessel. In an embodiment of the invention, the connection interface is flush mounted to a wall of a vessel. In one or more embodiments, a plurality of connection interfaces abuts a vessel and/or device. In an embodiment of the invention, a plurality of connection interfaces is surface mounted to a vessel. In an embodiment of the invention, a plurality of connection interfaces is surface mounted to a at least one wall of a vessel. In an embodiment of the invention, a plurality of connection interfaces is surface mounted to a vessel. In an embodiment of the invention, a plurality of connection interfaces is flush mounted to a vessel. In an embodiment of the invention, a plurality of connection interface is flush mounted to at least one wall of a vessel. In one or more embodiments, a plurality of connection interfaces is attached to a vessel, a container, and/or a device.

In an embodiment of the invention, the present invention discloses a first connection interface and a second connection interface. The first connection interface and the second connection interface may be similar or may be different in size and/or shape. In one or more embodiments, the exterior surface area facing ambient air, of the connection interfaces may be equal. In one or more embodiments, the exterior surface area facing ambient air, of the connection interfaces may be non-equal. In one or more embodiments, the difference in surface area between the exterior surface area of a plurality of connection interfaces is less than 0.3 inches squared. For example, less than 0.2 inches squared, or less than 0.1 inches squared.

In an embodiment of the invention, the first connection interface and the second connection interface when engaged with each other entrap ambient air particles therebetween. In an embodiment of the invention, the first connection interface and the second connection interface when engaged with each other externally displace from a vessel(s), and/or from the connection interface(s). In an embodiment of the invention, the first connection interface and the second connection interface when engaged with each other displace internally into an internal compartment of the vessel(s). In an embodiment of the invention, following an engagement between two vessels, the first connection interface displaces internally into an internal compartment of the vessel and the second connection interface displaces externally. In an embodiment of the invention, following an engagement between two vessels, the first connection interface and the second connection interface both displace internally into an internal compartment of one of the vessels. In an embodiment of the invention, following an engagement between two vessels, the first connection interface and the second connection interface both displace internally, wherein each displaces to its own vessel. In an embodiment of the invention, the first connection interface and the second connection interface include a sterile internal portion disposed in the first and second vessel, respectively, and an externally exposed portion, wherein when engaged with each other, the exposed portions displace externally and replaced by the internal sterile portions. In an embodiment of the invention, the first connection interface and the second connection interface include a sterile internal portion disposed in the first and second vessel, respectively, and an externally exposed portion, wherein when engaged with each other, the exposed portions displace internally into the vessel and replaced by the internal sterile portions.

In one or more embodiments, the connection interface is a thin film. In one or more embodiments, the connection interface has a thin film layer. In an exemplary embodiment, the connection interface may present a thickness of about 1 mm or below. Alternatively, the connection interface may present a thickness of above 1 mm. The connection interface may be manufactured from various materials, including, an elastic polymer. The connection interface may be made from a plastic material. The connection interface may be a rubber or a polymer. The connection interface may be formed of a metal material (e.g., a metal alloy). In yet a further exemplary embodiment, the connection interface may preset the form of a thin film made of an aluminum foil, cling wrap, or the alike. The metal alloy may include, for example, aluminum. In yet a further exemplary embodiment, the connection interface may preset the form of a thin film made of an aluminum foil, or the alike. In one or more embodiments, the connection interface is attached to, or is integrally formed with a vessel, or port thereof. In one or more embodiments, the connection interface is peelable from the vessels. In one or more embodiments, the peelable connection interface is attached to a vessel via an adhesive. In one or more embodiments, the peelable connection interface may be detached from a vessel surface by the force of a user's hand. In one or more embodiments, the connection interface may be attached to the vessel via a frangible seal that is breakable by the force of a user's hand. In one or more embodiments, the connection interface is movable or slidable along a rail located along a length of the aperture of a vessel. In one or more embodiments, the first and second connection interfaces may be attached to or engage each other via various mechanisms. For example, the connection interfaces may be engaged via an engagement mechanism, such as a thread, a luer, an adhesive, a mechanical object, a dent and a ratchet teeth mechanism. In one or more embodiments, the connection interface may be covered by a cover (not shown). In one or more embodiments, the cover may be a peelable cover. In one or more embodiments, a sterilizing or disinfecting substance may be located between the connection interface and the cover. In one or more embodiments, the sterilizing or disinfecting substance may be an alcohol. In one or more embodiments, the alcohol may be 70% isopropyl alcohol.

The connection interfaces may be coupled within any portion of the vessel. For example, the connection interfaces may be connected onto the vessels' body or aperture, the vessels' wall or port thereof. In one or more embodiments, at least a portion of the connection interface is disposed within a compartment of the vessel. Optionally, the vessel includes a fluid compartment for accommodating a medical substance and a fluid-free compartment and the connection interface or a portion thereof is disposed in the fluid-free compartment.

In one or more embodiments, the first connection interface and the second connection interface are configured to prevent contaminants from entering the fluid passageway between a first vessel and a second vessel. In one or more embodiments, the first connection interface and the second connection interface are configured to prevent contaminants from entering the interior of the vessel's body. In one or more embodiments, the first connection interface and the second connection interface are configured to prevent contaminants from entering a fluid housed inside the vessel's body.

In one or more embodiments, a first connection interface and a second connection interface are configured to prevent contaminants from contacting an internal compartment of the first vessel and/or the second vessel.

As used herein the term “external displacement” refers to a displacement (i.e., dislocation) of the herein disclosed first and/or second, and/or any additional connection interfaces. In an embodiment of the invention, the displacement is external, namely, outside the vessels being connected by the herein disclosed system. In an embodiment of the invention, the displacement is external to the fluid communication established following engagement of the vessels being connected by the herein disclosed system, containers, methods and devices. In an embodiment of the invention, the displacement maintains a hermetic seal of the connection interfaces and/or the vessels. The displacement may occur via a sliding motion, or by a pulling out motion, or by peeling the connection interfaces, or by a rotation motion. In an embodiment of the invention, the displacement occurs for one of the first connection interface and the second connection interface. In an embodiment of the invention, the displacement occurs for both the first connection interface and the second connection interface. The external displacement may optionally occur simultaneously for both connection interfaces or may occur consecutively. In an embodiment of the invention, the external displacement establishes a contaminant-free site where fluid communication may occur between vessels. In an embodiment of the invention, the external displacement establishes a fluid passageway between two or more vessels. In an embodiment of the invention, the external displacement establishes a sterile fluid passageway between two or more vessels. In one or more embodiments, the connection interface is configured to hermetically seal an aperture present in a vessel. In one or more embodiments, at about the time of external displacement of the connection interfaces, the aperture through which the connection interfaces pass through reseals, allowing a hermetic airtight connection between two or more vessels. In one or more embodiments, after external displacment of the connection interfaces, the aperture through which the connection interfaces pass through reseals, allowing a hermetic airtight connection between two or more vessels. In one or more embodiments, the aperture through which the connection interfaces pass through reseals prior to detachment of the connection interfaces from the vessels.

As used herein the term “internal displacement” refers to a displacement (i.e., dislocation) of the herein disclosed first and/or second, and/or any additional connection interfaces, and/or a portion thereof. In an embodiment of the invention, the displacement is internal, namely, into the vessel being connected by the herein disclosed system. In an embodiment of the invention, the displacement is internal, into a dedicated compartment within the vessel. In an embodiment of the invention, the displacement is internal, into a fluid-free compartment within the vessel. In an embodiment of the invention, the internal displacement does not hinder the fluid communication established following engagement of the vessels. In an embodiment of the invention, the displacement maintains a hermetic seal of the connection interfaces and/or the vessels. The displacement may occur via a sliding motion, or by a pulling out motion, or by peeling the connection interfaces, or by a rotation motion. In an embodiment of the invention, the displacement occurs for one of the first connection interface and the second connection interface. In an embodiment of the invention, the displacement occurs for both the first connection interface and the second connection interface. The internal displacement may optionally occur simultaneously for both connection interfaces or may occur consecutively. In an embodiment of the invention, the internal displacement establishes a fluid passageway between two or more vessels. In one or more embodiments, the connection interface is configured to hermetically seal an aperture present in/on a vessel. In one or more embodiments, at about the time of internal displacement of the connection interfaces, the aperture through which the connection interfaces pass through reseals, allowing a hermetic airtight connection between two or more vessels. In one or more embodiments, after internal displacement of the connection interfaces, the aperture through which the connection interfaces pass through reseals, allowing a hermetic airtight connection between two or more vessels. In one or more embodiments, the aperture through which the connection interfaces pass through reseals prior to detachment of the connection interfaces from the vessels.

As used herein the term “vessel” refers to any device utilized for containing, housing or transferring substances as herein disclosed. In one or more embodiments, the vessel may be used for containing medical substances. In an embodiment of the invention, the vessel is a medical vessel. In an embodiment of the invention, the vessel is a medical device. In an embodiment of the invention, the vessels are used for, and adapted to allow connection to another vessel(s). In an embodiment of the invention, the vessel may be a medical container utilized for accommodating medical substances. Various types of medical containers are contemplated. The medical container may be selected, without limitation, from a vial, a bag, a chamber, a housing, a bottle, and the alike. In an embodiment of the invention, the term vessel further encompasses elements that can be used to connect between vessels. In accordance with this embodiment, the vessel may be selected, without limitation, from a connector, a port, a syringe, an infusion line, a tubing, a syringe, a filter, a spike, a port and a manifold. In an embodiment of the invention, one or more connection interfaces (for example, two or more, three or more, etc.) may be coupled to a first vessel and each of those connection interfaces may be coupled and engaged with one or more connection interfaces present on one or more vessels. In one or more embodiments, the vessel may be a container, a bag, a bottle, a connector, an infusion line or a syringe having a plurality of externally displaceable connection interfaces. In one or more embodiments, the vessels to be engaged may have similar surface area or similar contact surface area (i.e., surface onto which the connection interface is coupled to). For example, a first vessel and a second vessel may have similar surface area or similar contact surface area. In one or more embodiments, the vessels to be engaged may have different surface area or different contact surface area. For example, a first connection interface may have a greater surface area or contact surface area than a second vessel. Optionally, the vessel contains two distinct compartments. Optionally, the vessel includes a first compartment for accommodating a medical substance and a second compartment for accommodating the connection interface. Optionally, the second compartment is a fluid-free compartment. Optionally, the interior of the vessel is sterile. Optionally, the second compartment is a sterile compartment.

As used herein the term “fluid communication” refers to two vessels in which substances may pass therethrough either directly or indirectly. The fluid communication may occur via a fluid passageway that allows for the flow/transfer of a medical substance.

The herein disclosed systems may optionally include a mechanism for allowing the movement of the connection interfaces. The movement mechanism may be a rotating mechanism, such as a wheel, which is disposed within the vessel, optionally both vessels of a system. The rotating mechanism may be operable to allow at least one of: displacement (internal or external) of the connection interface, and fluid passageway by accessing a conduit crossing the rotating mechanism to an aperture of the vessel. the rotating mechanism may rotate in a clockwise direction and/or a counterclockwise direction. Optionally, the rotating mechanism rotates within the vessel to allow displacement and/or fluid passageway by the conduit and thereafter rotates backwards to its initial position, optionally, to seal an aperture of the vessel. In one or more embodiments, the system includes two vessels, wherein at least one of the vessels includes a rotating mechanism. In one or more embodiments, the rotating mechanism of one vessel forcibly rotates a rotating mechanism of another vessel. The forcible rotation may be due to a force of friction. In one or more embodiments, each rotating mechanism in each vessel operates independently from the other vessel. Optionally, the rotating mechanism includes a valve, optionally disposed in a conduit crossing the rotating mechanism. The valve configured to control or restrict the fluid passage between vessels. Other valve locations may be applicable. For example, one or more valves may be located at an aperture of one or more vessels. In a further exemplary embodiment, one or more valves may be disposed at the aperture(s) of the vessels or at a location within a port or vessel body of the vessels. Optionally, the valve is disposed in the fluid-free compartment of the vessel. The valves are generally provided to seal and prevent fluid flow prior to any engagement of vessels and/or external and/or an internal displacement of connection interfaces. In an exemplary embodiment, when valves are provided to seal an aperture that provides for the displacement of connection interfaces, the valves may establish resealing of the apertures at about the time or following the displacement of the connection interfaces, preventing any leakage of fluids from the engagement between the vessels. In certain embodiments, the resealing occurs at about the time or prior to the displacement. In certain embodiments, the valves that provide for the passage and displacement of the connection interfaces may be made of a rubber material, an elastomeric material, a plastic material, and combinations thereof In an embodiment of the invention, the valve or aperture providing for the passage and displacement of at least one or two connection interfaces maintains a hermetic seal between at least two vessels or at least two devices by a force of the engagement between at least two vessels or devices. In some embodiments, the force of engagement that maintains the hermetic seal between two vessels or devices during the displacement of the connection interfaces is provided by an engagement mechanism selected from a thread, a luer, a ratchet teeth mechanism, a snap-on mechanism, and combinations thereof. In certain embodiments, the aperture, sealed aperture, or valve providing for the passage and displacement of at least one or at least two connection interfaces reseal prior to the connection interfaces fully displacing from the first and/or second vessel or device. In certain embodiments, the valves may prevent backflow of fluids. In some embodiments, the valves may be one-way valves. In one or more embodiments, the movement of the connection interfaces is provided by a rail mechanism on at least one of the first and second vessels, the rail mechanism configured to provide the displacement of the connection interface. In one or more embodiments, the first connection interface and/or the second connection interface are externally displaced via a sliding motion. In one or more embodiments, the first connection interface and the second connection interface displace at, or about the same time.

Optionally, the vessels and/or ports of the systems are provided with seals/covers and those seals/cover become open upon piercing by a piercing member or breaking/rupturing thereof. Optionally, the vessels and/or ports of the systems are provided with seals/covers and those seals/covers are opened upon uncovering or removing thereof by a user. Optionally, one or more of the seals/covers disclosed herein are fragile, allowing piercing thereof with a sharp instrument (i.e., a piercing member) or by the application of a sufficient force to break or rupture the seals/covers. The piercing member may optionally be a needle, for example, a hollowed needle, but other sharp elements are contemplated.

In an embodiment of the invention, the herein disclosed devices, systems and methods allow fluid communication in contaminant-free, or substantially contaminant-free manner. For example, between three or more, four or more, five or more or six or more vessels. In an embodiment of the invention, the herein disclosed devices, systems and methods allow fluid communication between three, four, five, six, or seven vessels. For example, the devices, systems, and methods of the invention allow fluid communication between two containers, between two bags, between a syringe and a bag, between a connector and a bag, between a vial and a bag, between a vial and a bottle, between a syringe and a vial or between a syringe and a connector.

A further purpose of this invention is to provide a connection interface that reduces the presence of non-purified air and/or air particles. This invention focuses on connection interfaces that are substantially reduced of contaminants and ambient air particles and/or entirely contaminant free and entirely free of ambient air particles.

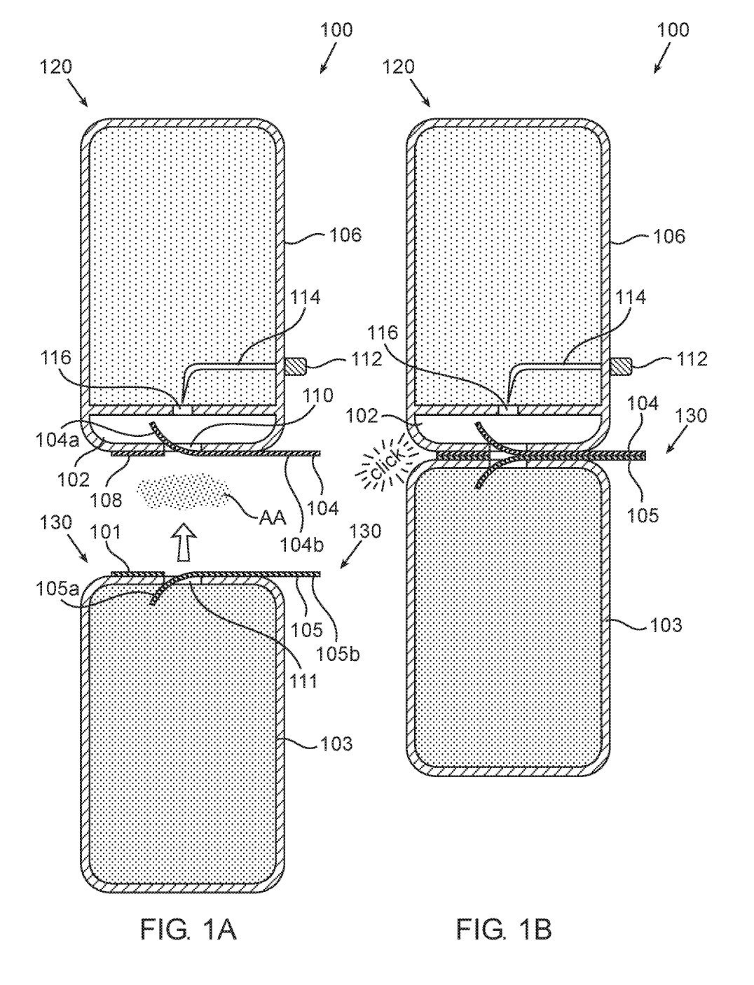

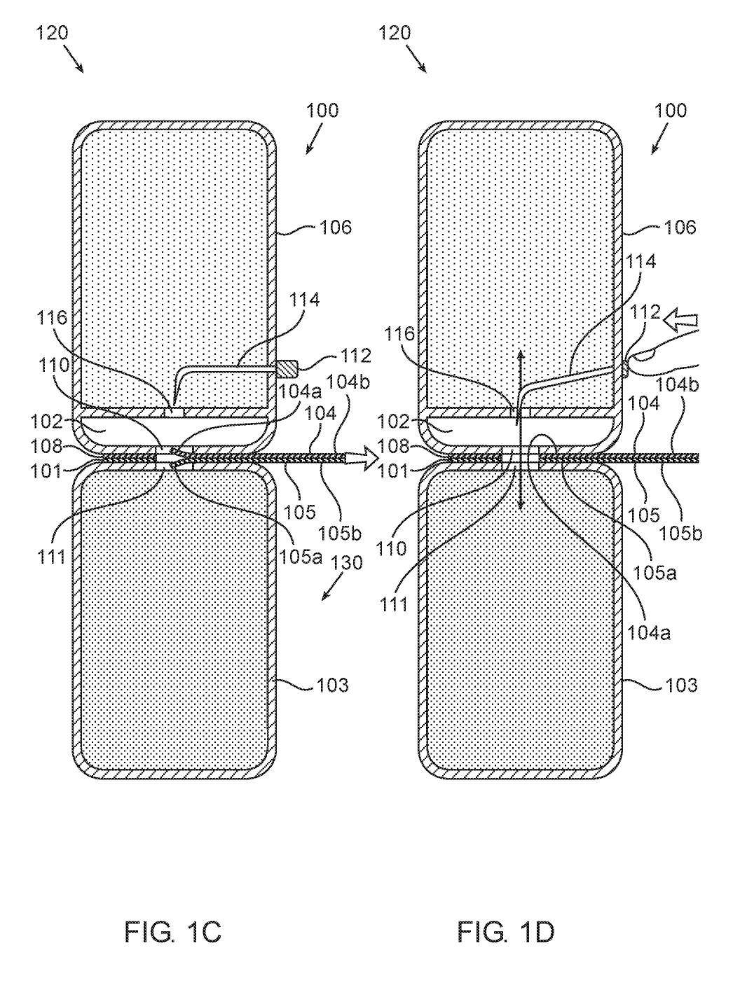

Referring now to the drawings, FIGS. 1A-1D illustrate an exemplary decontamination system 100 for the engagement and fluid transfer of one or more vessels in a decontaminated manner. The system 100 includes a first vessel 120 and a second vessel 130, the vessels configured to engage with each other via a first connection interface 104 coupled to first vessel 120 and a second connection interface 105 coupled to second vessel 130. As herein demonstrated, first vessel 120 includes two compartments, being a fluid compartment 106 which is configured to accommodate a medical substance and a fluid-free compartment 102 which is substantially dry. Second vessel 130 includes a single fluid compartment 103 configured to accommodate a medical substance. First connection interface 104 is composed of a sterile, internal portion 104a concealed within fluid-free compartment 102 and a non-sterile, externally exposed portion 104b. Second connection interface 105 is composed of a sterile, internal portion 105a concealed within vessel 130 and a non-sterile, externally exposed portion 105b. Those external portions 104b and 105b continuously extend from the internal portions 104a and 105a, respectively, to the environment, and are exposed to ambient air (AA) particles. Vessels 120 and 130 can engage with each other via the exposed portions of the connection interfaces 104b and 105b and the engagement may be optionally secured by an engagement mechanism in the form of adhesives 101 and 108. Optionally, second vessel 130 may further comprise a fluid-free compartment (not shown) similar to first vessel 120 in which internal portion 105a may reside.

FIG. 1A illustrates the vessels 120 and 130 when spaced apart and AA surround them. FIG. 1B illustrates the vessels 120 and 130 following an engagement therebetween the vessels, optionally, via the adhesives 101 and 108. AA may be entrapped by the engaged externally exposed interfaces 104b and 105b. At FIG. 1C, an external displacement of the connection interfaces 104 and 105 is shown. Optionally, the entire portions of one or both connection interfaces 104 and 105 displace externally, and away from the engaged vessels 120 and 130, maintaining a decontaminated engagement between the vessels 120 and 130. Alternatively, the exposed portions 104b and 105b of the connection interfaces displace externally from the vessels' engagement and replaced by the internal sterile portions, maintaining a sterile engagement between the vessels via the sterile portions 104a and 105a of the connection interfaces 104 and 105, respectively. A piercing member 114 can then be actuated by a user, optionally, via pushing button 112, to pierce a seal (not shown) sealing aperture 116 of the fluid compartment 106. The openings 110 and 111 of vessels 120 and 130 which were initially covered by the connection interfaces 104 and 105 may be made open at the time of displacement of the connection interfaces 104 and 105, respectively. A fluid passageway via internal and external openings 116 and 110 of vessel 120 and aperture 111 of vessel 130 can be established allowing fluid transfer or exchange. Piercing member 114 may pierce any, or all, of openings 116, 110, and 111.

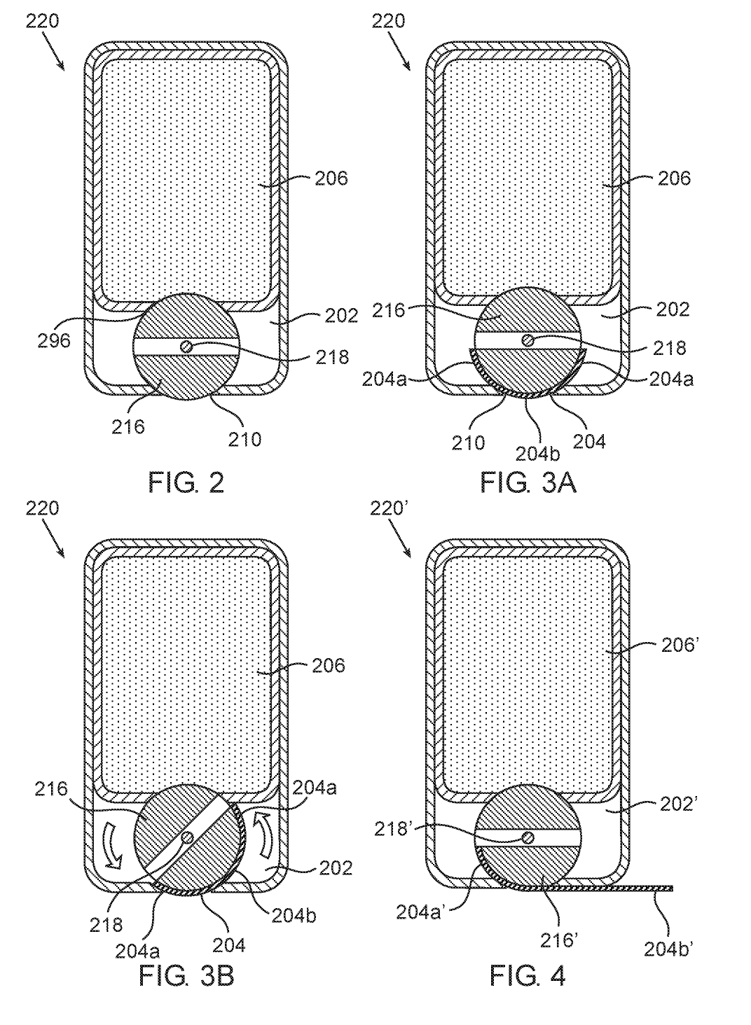

FIG. 2 illustrates an exemplary vessel 220 which includes a fluid compartment 206 and a fluid-free, or a substantially dry compartment 202. A rotating mechanism in the form of wheel 216 is provided to allow for the displacement of a connection interface via rotation of wheel 216. Optionally, wheel 216 blocks openings 210 and/or 296 of vessel 220. Optionally, the rotation of wheel 216 within vessel 220 is via hinge 218.

FIGS. 3A-3B illustrate vessel 220 which further includes a connection interface 204 that at least partially surrounds rotating mechanism 216. Both ends 204a of the connection interface 204 are sterile and concealed within fluid-free compartment 202 of vessel 220. A middle portion 204b is exposed to the environment and therefore non-sterile. An internal displacement of the exposed portion 204b is shown in FIG. 3B via rotation of wheel 216. The sterile portion 204a replaces the exposed portion 204b, such that a second vessel (not shown) may be engaged with vessel 220 via the sterile portion 204a. The rotating mechanism 216 is shown in FIG. 3B when rotating counterclockwise, but a clock wise rotation is also applicable and herein contemplated.

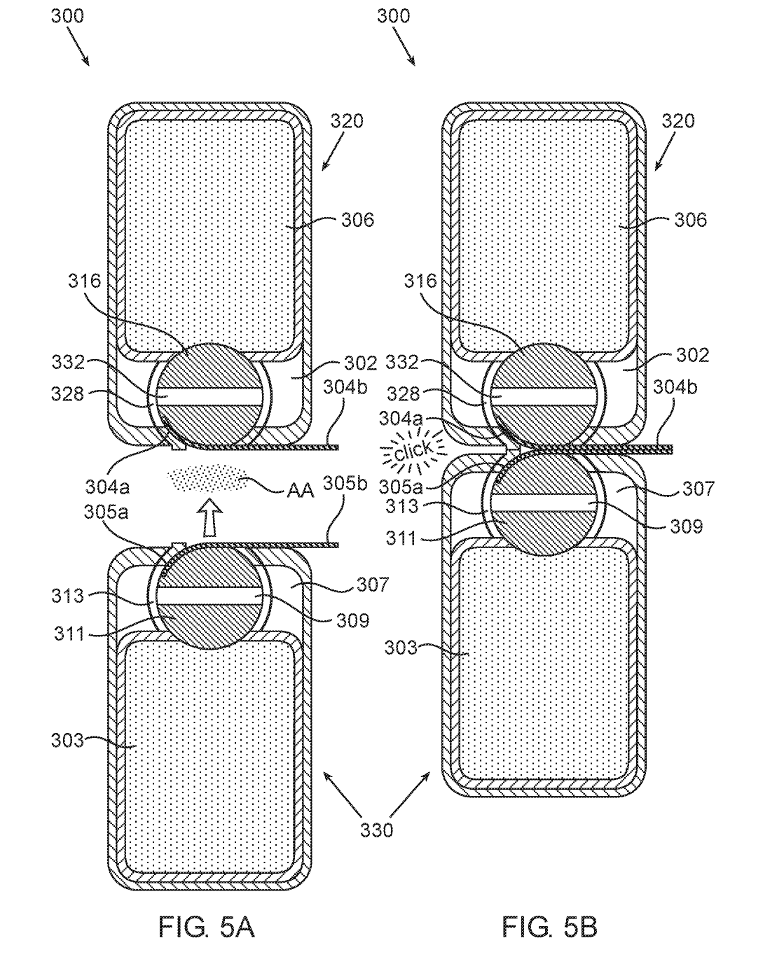

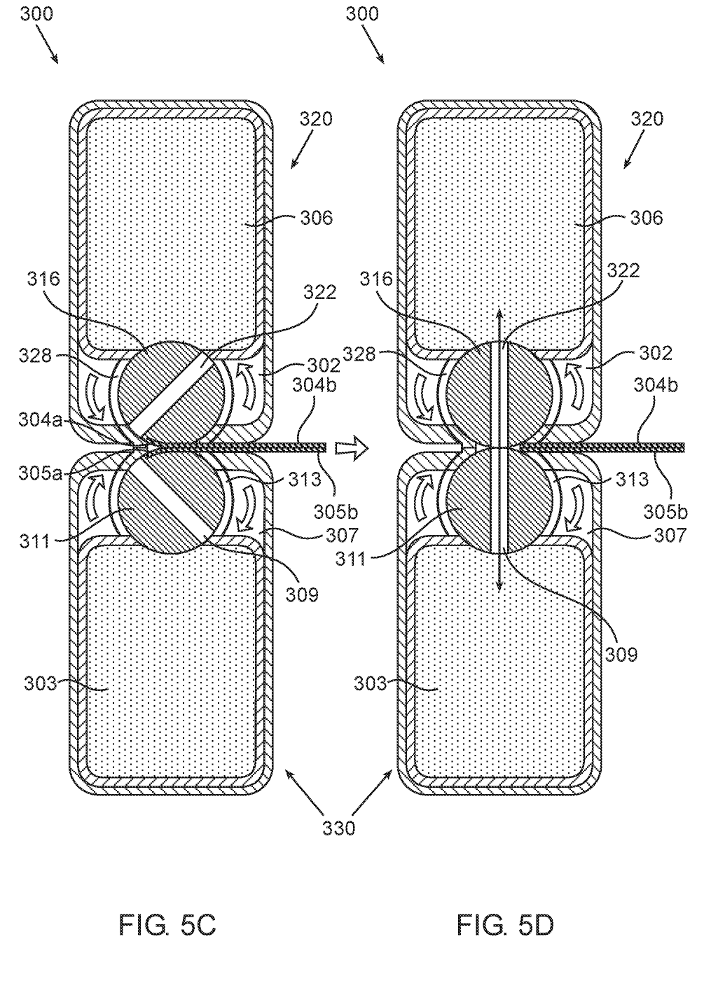

FIG. 4 illustrates yet a further exemplary arrangement of connection interface 204′ in vessel 220′. Similarly to connection interface 104 of vessel 120, the connection interface 204′ includes a sterile portion 204a′ concealed in compartment 202′ and a non-sterile exposed portion 204b′ which continuously extends from portion 204a′. The following system 300 demonstrated in FIGS. 5A-5D shows an engagement and fluid passageway of vessels having similar design of connection interfaces.

FIGS. 5A-5D illustrate system 300 which includes first vessel 320 and second vessel 330. Both first and second vessels 320 and 330 include a fluid compartment 306 and 303, and a fluid-free compartment 302 and 307, respectively. A sterile portion of the connection interface 304a resides within the fluid-free compartment 302, optionally within a dedicated channel 328 surrounding rotating mechanism 316. Similarly, a sterile portion of the connection interface 305a resides within the fluid-free compartment 307, optionally within a dedicated channel 313 surrounding rotating mechanism 311. At FIG. 5A, the vessels 320 and 330 are shown when spaced apart and AA surround the interfaces 304 and 305. At FIG. 5B, the vessels engage with each other via the exposed portions 304b and 305b of the connection interfaces 304 and 305, respectively. An audible sound, optionally a “click” sound may be emitted at the time of engagement between the vessels 320 and 330. The external displacement of the interfaces 304 and/or 305 may be effected via pulling out the exposed edges of the interfaces and/or by rotating the wheel 311 and/or 316 (FIG. 5C). Rotating mechanism 316 is rotated about 90° counter clock-wise and rotating mechanism 311 is rotated about 90° clock-wise, thereby forming a fluid passageway between the vessels via conduits 322 and 309 which now disposed in a vertical position, forming a connection path between the fluid compartments of the vessels 320 and 330 (FIG. 5D). 332

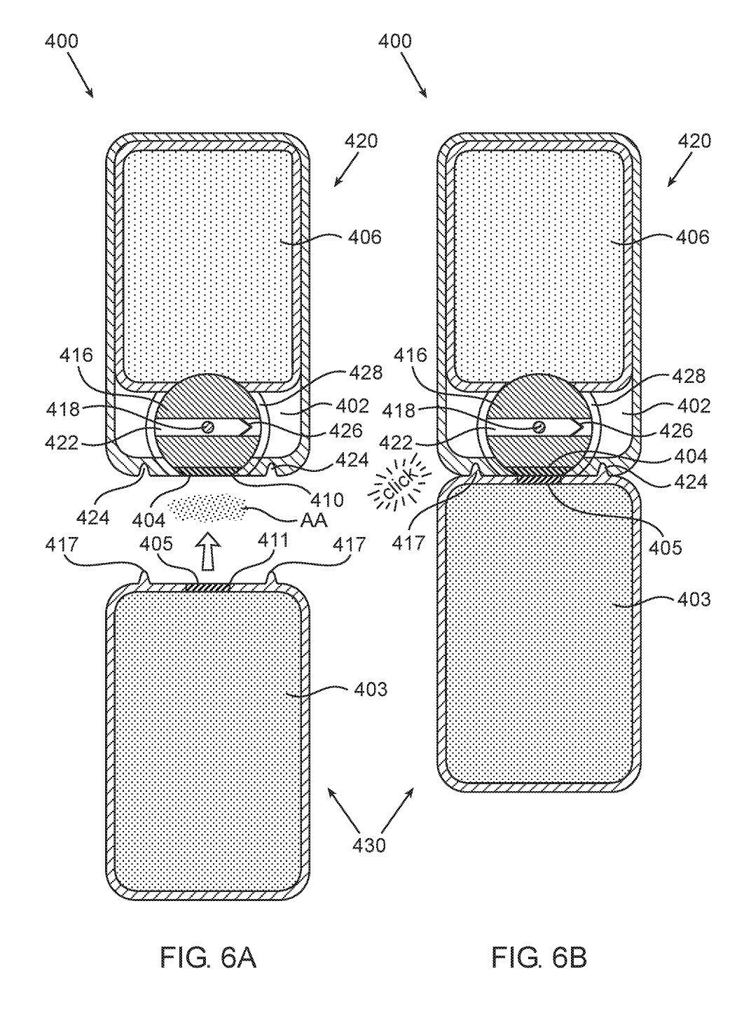

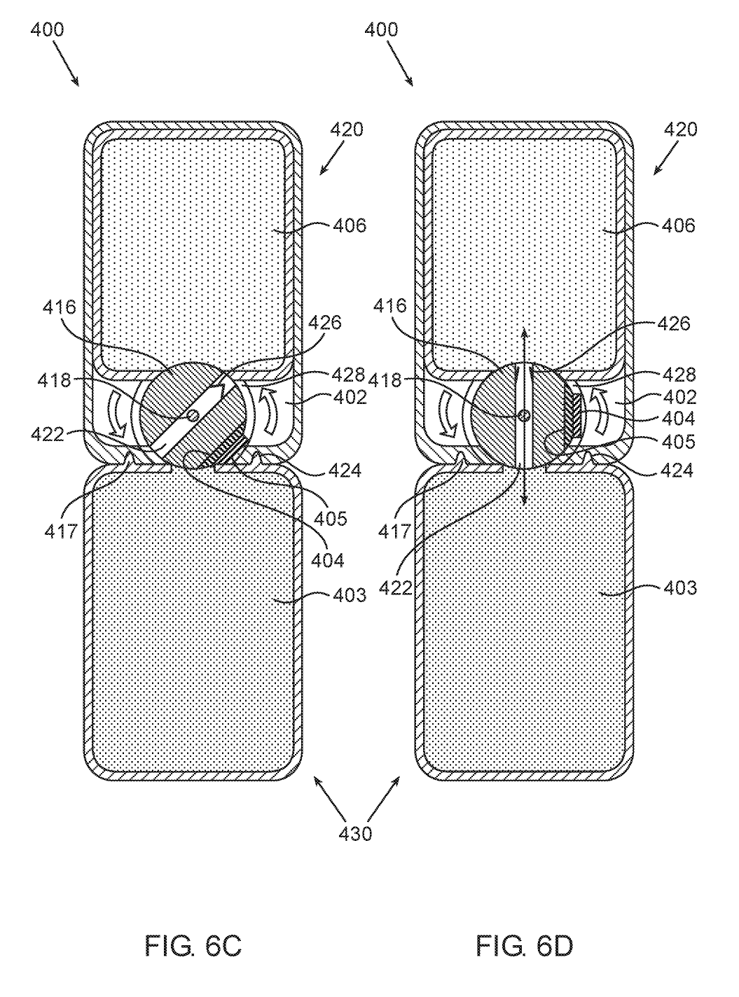

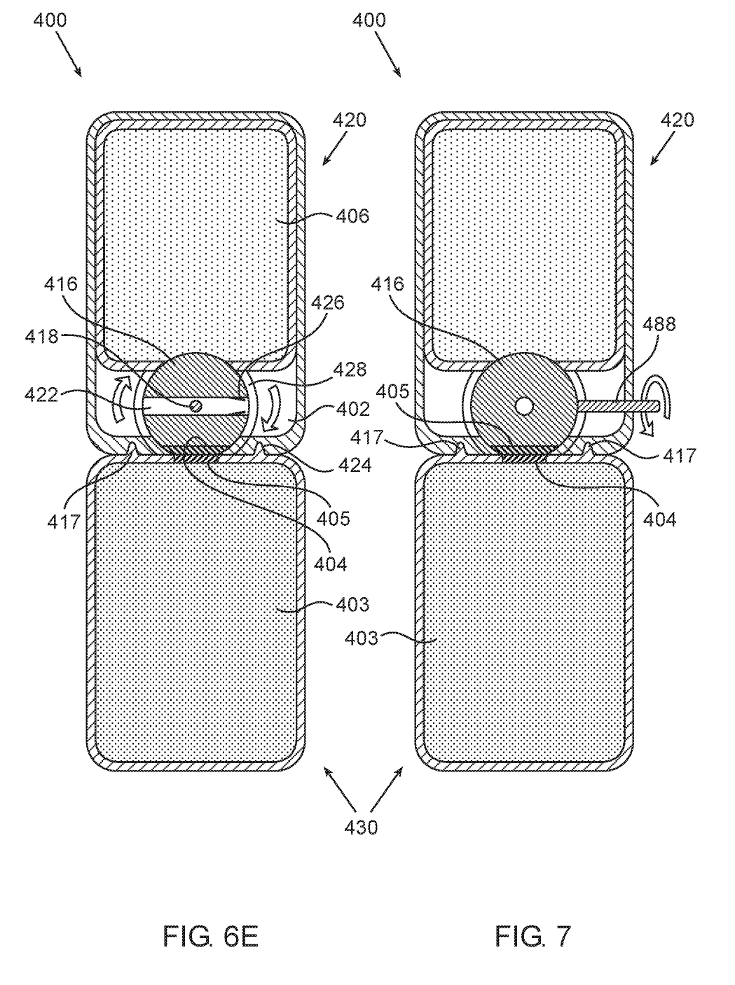

FIGS. 6A-6E illustrate system 400 which includes first vessel 420 and second vessel 430 and a rotating mechanism in the form of wheel 416 disposed on/in first vessel 420. In first vessel 420, connection interface 404 is fully exposed to AA and seals aperture 410. In second vessel 430, connection interface 405 is fully exposed to AA and seals aperture 411. Vessel 430 includes one fluid compartment 403. Vessel 420 includes a fluid compartment 406, a fluid free compartment 402 and a rotating mechanism 416. The system 400 includes an engagement mechanism in the form of dents 424 and teeth 417 which can engage with each other, optionally lock into each other, optionally in a permanent manner. Once engaged. (FIG. 6B), rotating mechanism 416 can be rotated, thereby internally displacing both interfaces 404 and 405 into vessel 420. The interfaces are displaced into fluid-free compartment 402, optionally within an internal channel 428 surrounding rotating mechanism 416 (FIG. 6D). Following the rotation of the rotating mechanism 416, valve 426 is opened and a fluid passageway may be formed between vessels 420 and 430 via conduit 422. As shown in FIG. 6E, the rotator 416 can be rotated to its initial position, returning the interfaces 404 and 405 into their initial or original positions, thereby resealing the openings 410 and 411 of the vessels. A side view of system 400 demonstrates a handle 488 in FIG. 7 which extends from an opening in a side wall of the vessel and which allows a user to rotate the wheel 416.

FIG. 7 is a side view illustration of the system of FIGS. 6A-6E, showing rotating mechanism 416 and handle 488, as well as the connection of interfaces 404 and 405.

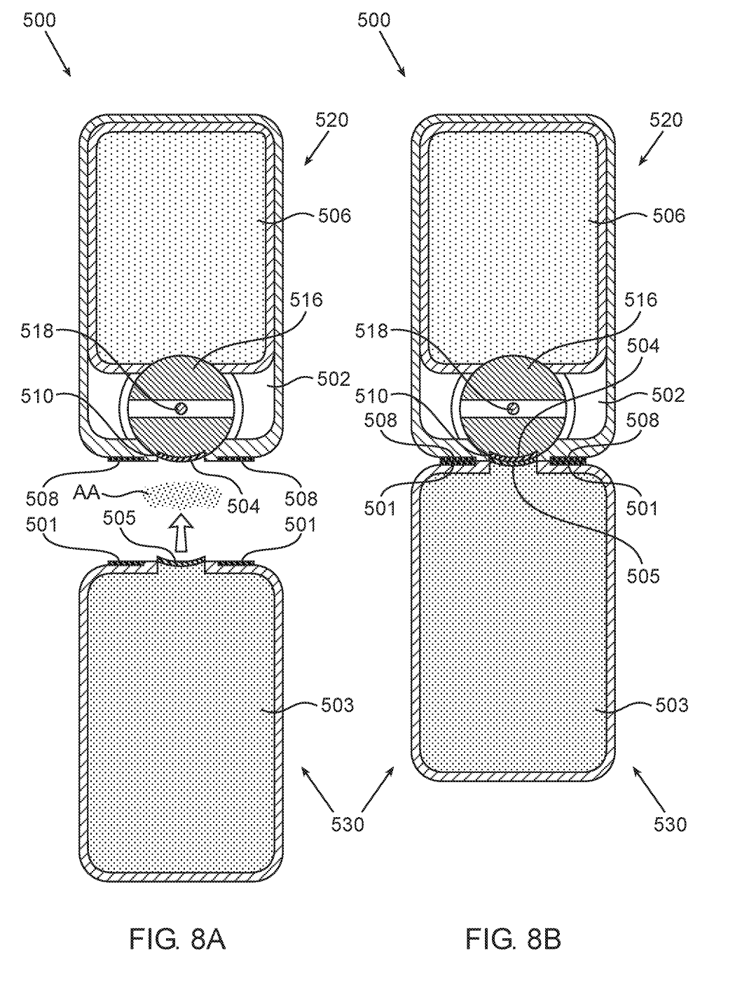

FIGS. 8A-8B illustrate a system 500 with an engagement mechanism in the form of adhesives 501 and 508, to allow for an engagement between two vessels 520 and 530.

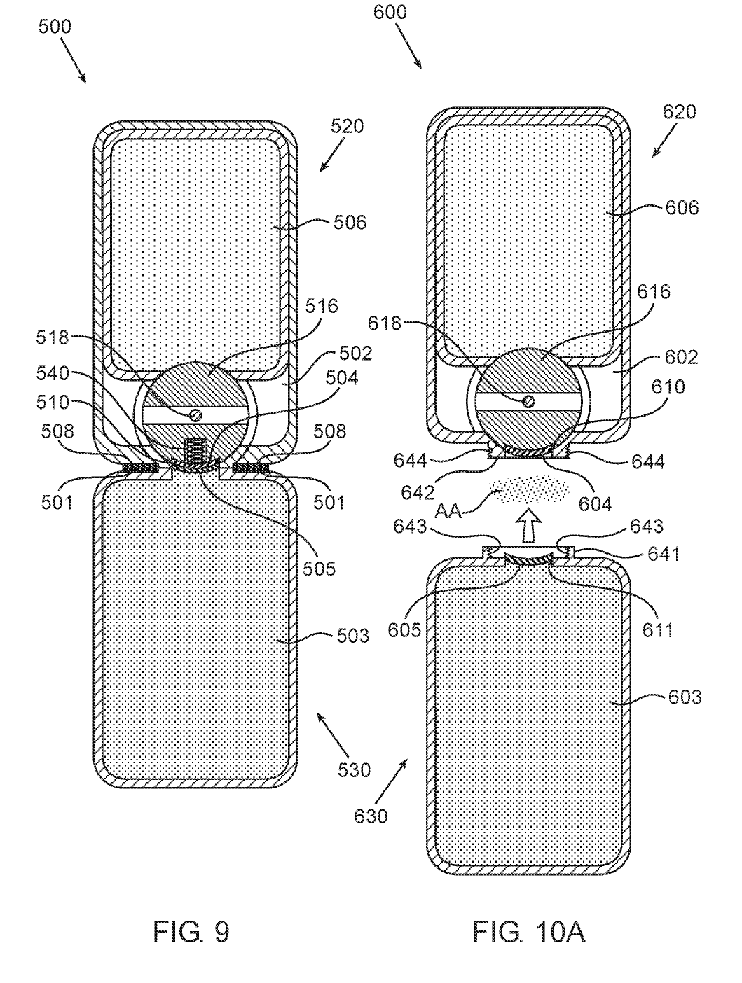

FIG. 9 is a cut view illustration of the system of FIGS. 8A-8B which further includes press mechanism 540 in the form of a spring which is provided to hermetically seal aperture 510, by pressing connection interface 504 from within the vessel 520 and against aperture 510. System 500 includes connection interfaces 504 and 505 which are exposed to AA. Adhesives 501 and 508 allow for a hermetic, optionally airtight seal connection between the vessels 520 and 530. Optionally, the hermetic, and/or airtight seal connection is maintained following the displacement of the interfaces, internally within vessel 520 and/or 530. The spring may be replaced with other types of press mechanisms such as rubber structures, elastomeric structures, and the such that will/may press connection interface 504 against aperture 510 to seal aperture 510 while also allowing displacement of connection interface 504 either internally or externally, optionally both internally and externally.

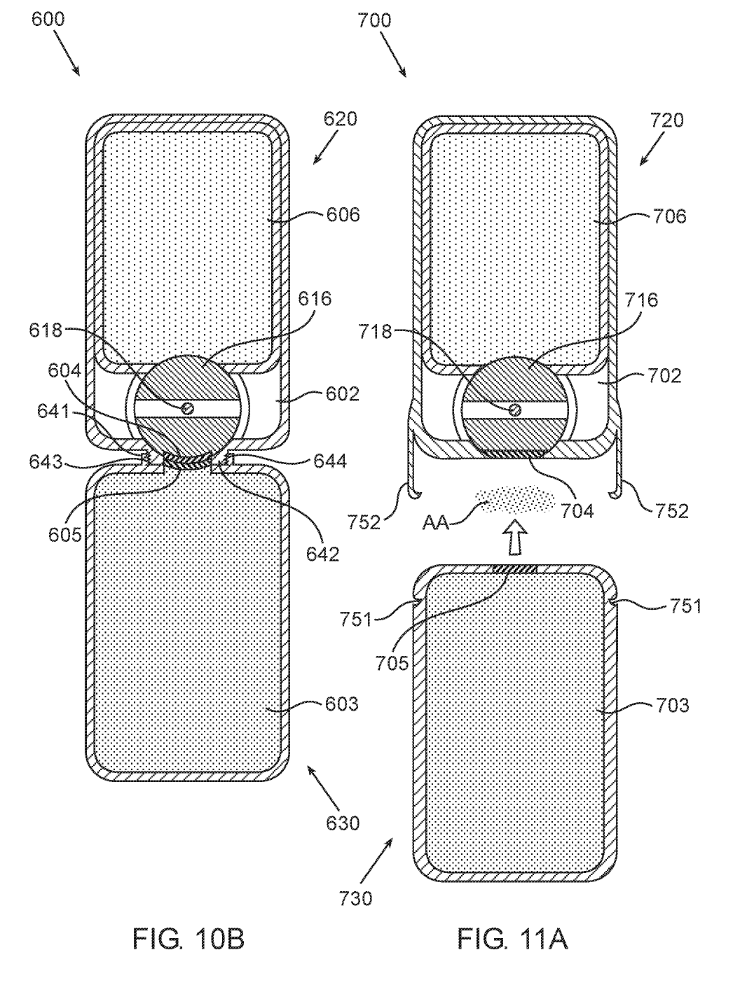

FIGS. 10A-10B demonstrate yet a further exemplary system 600 which includes a first vessel 620 and a second vessel 630. Vessel 620 includes side walls extending vertically from a bottom outer surface of the vessel and defining port 642. Vessel 630 includes side walls extending vertically from an upper outer surface of the vessel and defining port 641. Ports 642 and 641 include an engagement mechanism in the form of external thread 644 configured to cooperate with internal thread 643. Threads 643 and 644 optionally allow for a hermetic, optionally airtight seal connection between the vessels 620 and 630. Optionally, the hermetic, and/or airtight seal connection is maintained following the displacement of the interfaces, internally within vessel 620 and/or 630. As in the previous figures, connection interfaces 604 and 605 are further operable so as to seal apertures 610 and 611, such that following the displacement of the interfaces the apertures 610 and 611 are opened and a fluid passageway may be formed.

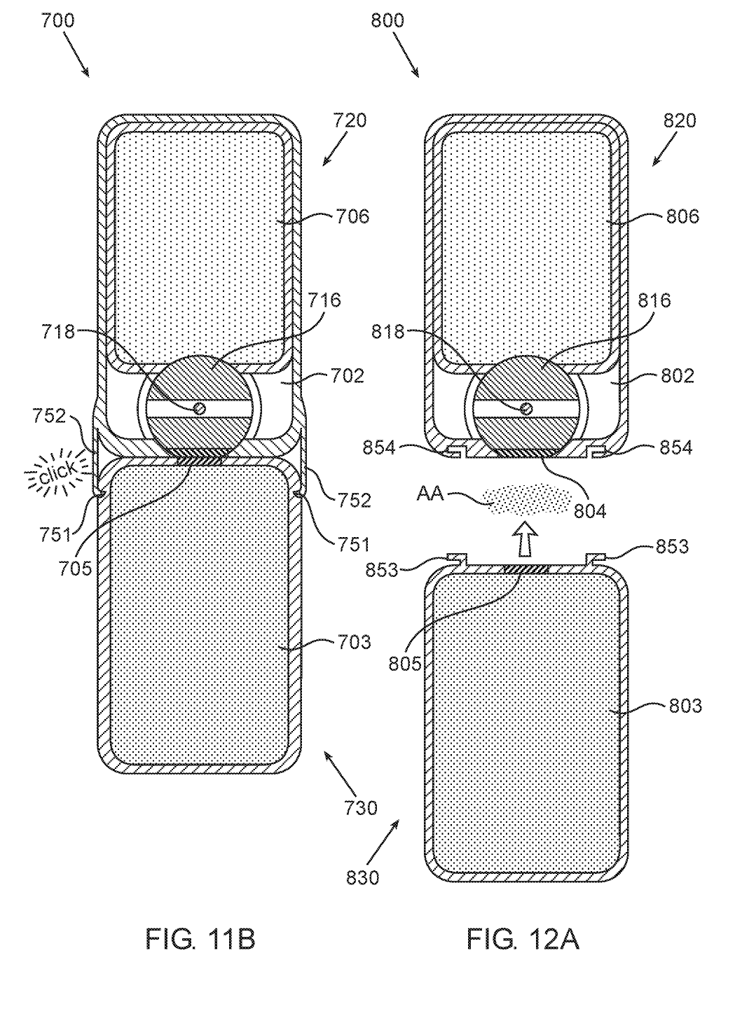

FIGS. 11A-11B demonstrate yet a further exemplary system 700 which includes, clamping arms 752 as the engagement mechanism. Clamping arms 752 are disposed to downwardly extend from opposing side walls of vessel 720. The arms 752 are configured to lock into or attach dents 751 on opposing side walls of vessel 730. Optionally, the engagement between the vessels is permanent and/or irreversible. Following and/or at the time of engagement of the vessels via clamping arms 752, the connection interfaces 704 and 705 attach to each other and thereafter displaced, allowing a fluid passageway between vessels 720 and 730. External surfaces of connection interfaces 704 and 705 may have an adhesive disposed on the external surfaces so that connection interfaces 704 and 705 attach to each other and afterwards displace simultaneously. The adhesive may be, in some embodiments, similar to a glue and/or tape like substance.

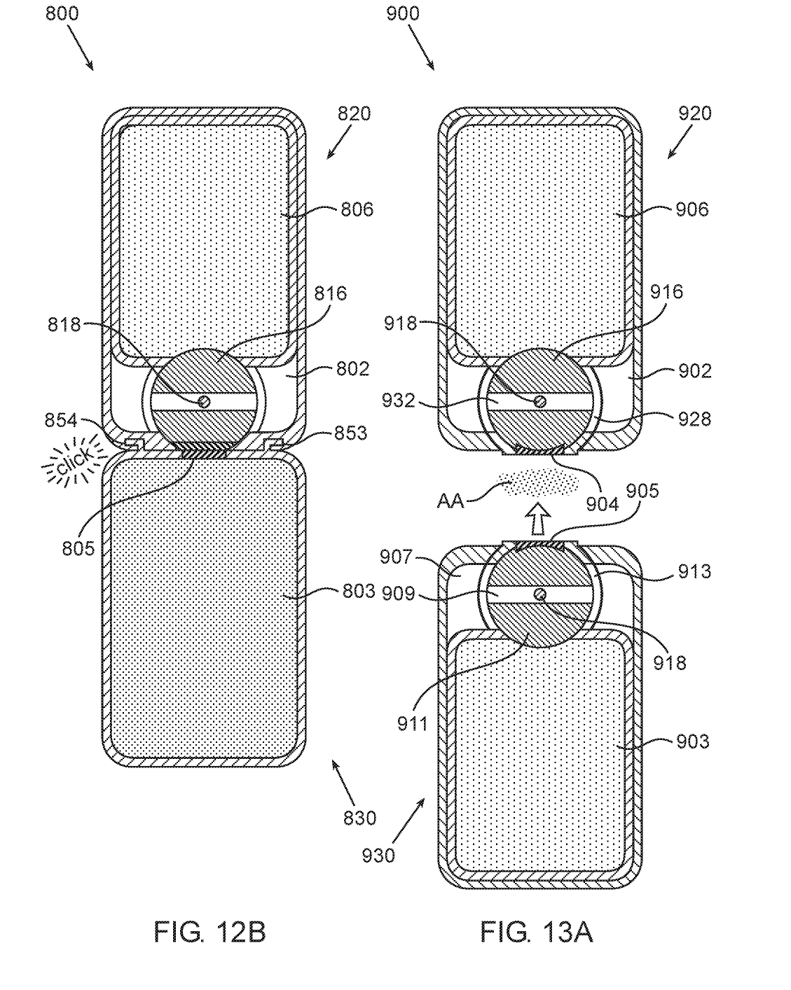

FIGS. 12A-12B demonstrate an engagement mechanism in the form of square hooks 853 which fit to lock into complementary dents 854 is shown in FIGS. 12A-12B. An audible sound, optionally a “click” sound, may be emitted at the time of engagement between the vessels 820 and 830. Similar to systems 400-700, system 800 includes a dual compartment first vessel 820 and a single compartment second vessel 830. A wheel 816 disposed in first vessel 820 is configured to rotate the interfaces 804 and 805 into the fluid free compartment 802 of first vessel 820. It is explicitly stated here that any of the systems and/or containers in the specification and/or any of the drawings may have dual compartment vessels. Any of the systems in the drawings where one dual compartment first vessel is shown may also have s second vessel having a dual compartment, even if it is not shown in the drawings.

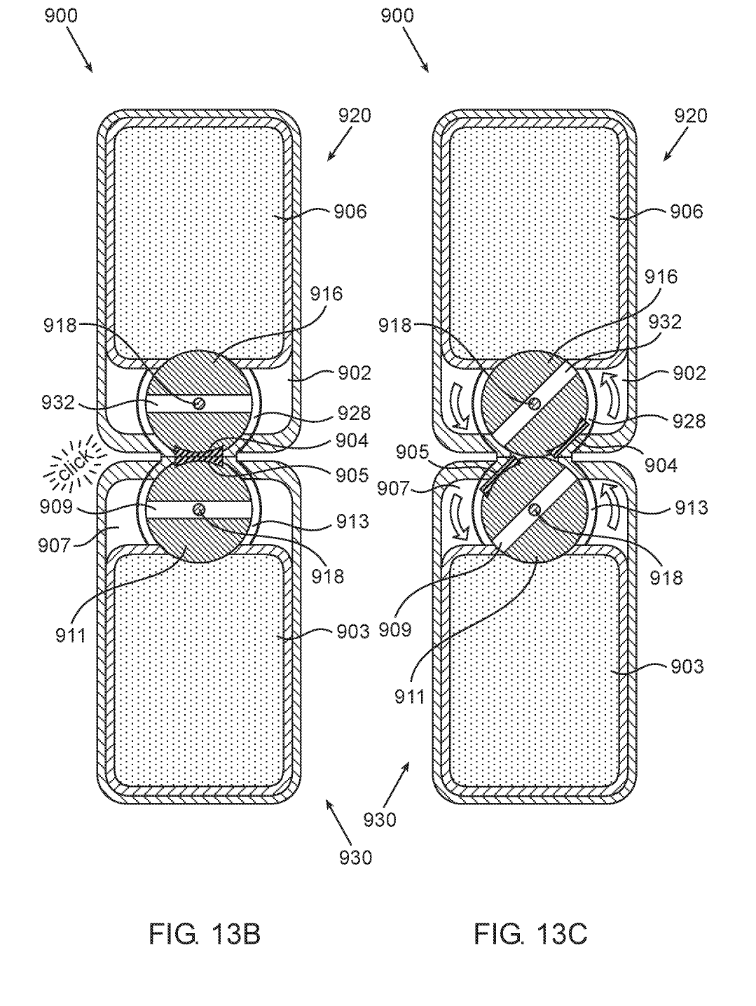

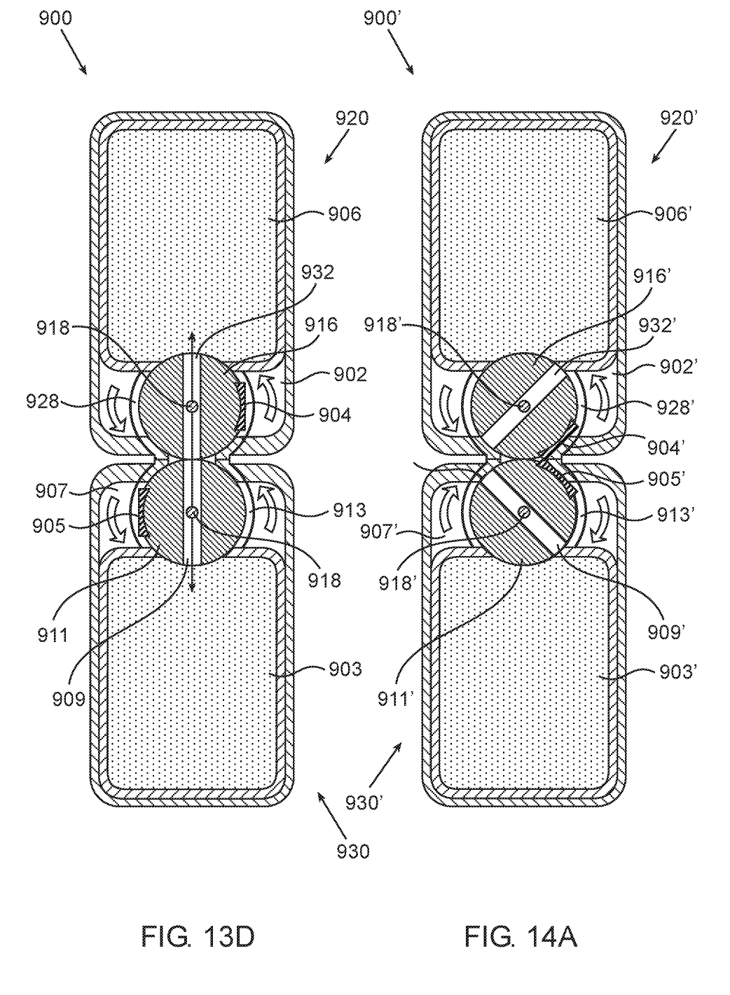

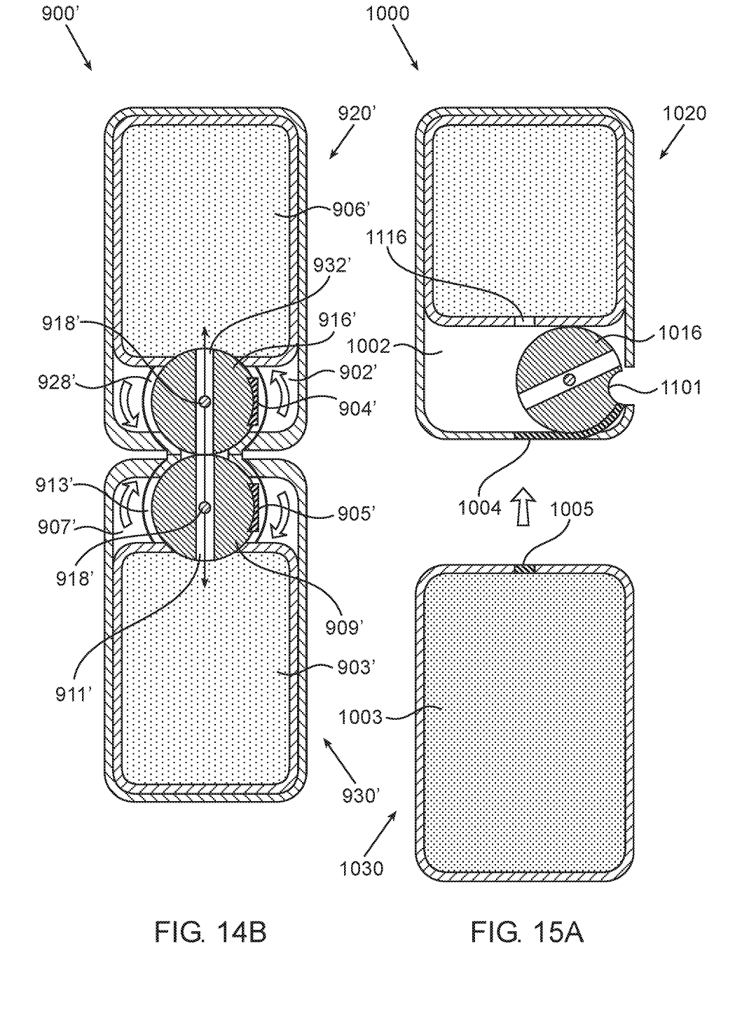

Systems 900 and 900′ demonstrated in FIGS. 13 and 14, each include two vessels, i.e., vessels 920, 930 and vessels 920′, 930′. Each of the vessels includes its own rotation mechanism in the form of wheels 916, 911, and 916′, 911′ and the connection interfaces 904, 905 and 904′, 905′ displace internally, each into its own vessel of origin. The rotating mechanisms in those systems may rotate independently by a user. Alternatively, rotation of one wheel automatically forces rotation of another wheel. System 900 demonstrates an internal displacement of connection interfaces 904 and 905 via rotation. Both wheels 911 and 916 rotate in a counter clock wise direction. System 900′ demonstrates an internal displacement of connection interfaces 904′ and 905′ via rotation, wherein first vessel 920′ rotates counter clock wise and second vessel 930′ rotates in a clock wise direction. Following about 90° rotation of the wheels 911 and 916, a fluid passageway may be formed via conduits 909 and 932 (FIG. 13D). Similarly, in system 900′, following about 90° rotation of the wheels 911 and 916, a fluid passageway may be formed via conduits 909′ and 932′ (FIG. 14B).

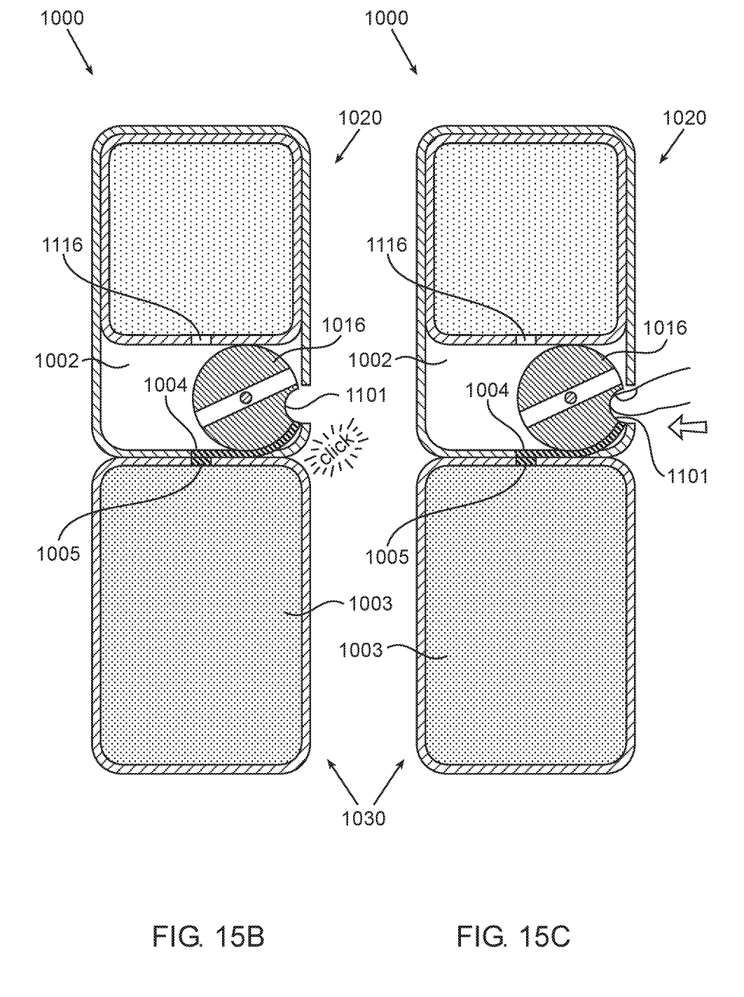

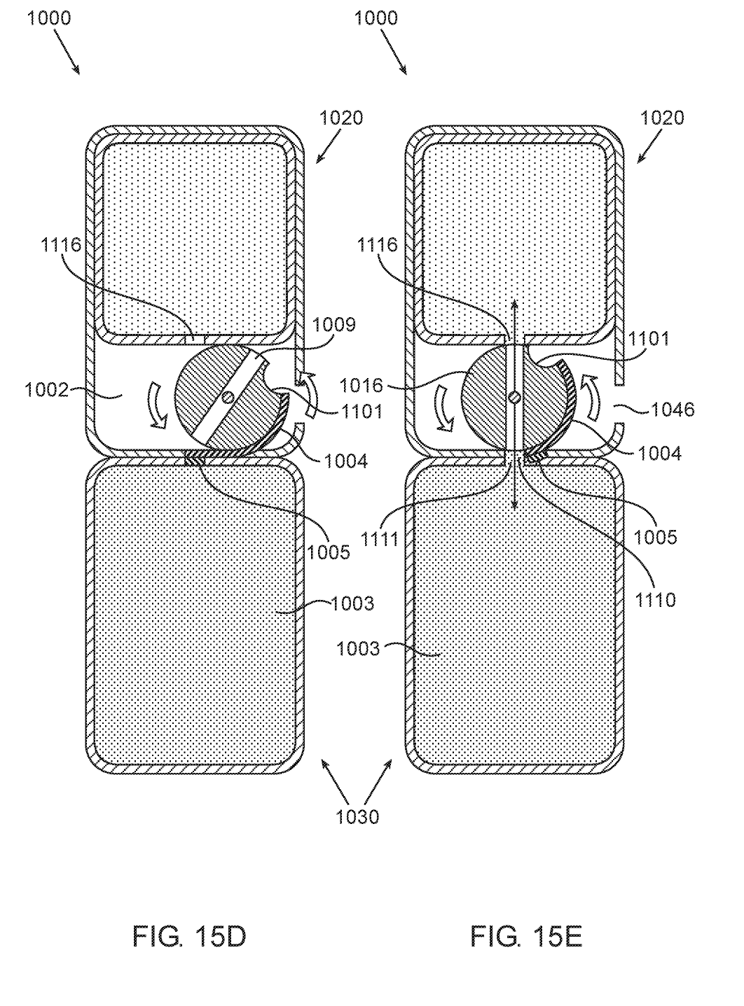

Referring now to FIGS. 15A-15E, a system 1000 is shown. Here, first vessel 1020 includes a rotating mechanism 1016 which disposed in fluid free compartment 1002, proximate a side wall of compartment 1002. The rotating mechanism includes a dent 1101 for operating the wheel 1016 from the outside via a dedicated opening 1046 in the side wall. The finger of a user may swipe the dent 1101 from the outside, thereby rotating the wheel 1016. The wheel 1016 rotates to vertically align the conduit 1009 such to create a path between apertures 1116, and 1110 of vessel 1020, and aperture 1111 of vessel 1030, thereby allowing a fluid passageway between the vessels 1020 and 1030. Upon rotation of wheel 1016, an internal displacement of interface 1005 within compartment 1002 occurs. In addition, the exposed portion of connection interface 1004 also displaces internally within compartment 1002. The apertures 1110, and 1111 which were initially sealed by the connection interfaces 1004 and 1005, respectively, are opened upon the displacement of the interface. Optionally, apertures 1110 and 1111 may remain sealed by a pierceable and/or frangible seal after displacement of connection interfaces 1004 and 1005.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for decontaminating a surface of vessels, the system comprising:
a first connection interface attached to a first vessel; and
a second connection interface attached to a second vessel,
wherein the first connection interface and the second connection interface having a portion that is externally exposed and a portion that is internally concealed within the vessel,
wherein said first vessel and said second vessel are configured to engage with each other, and
wherein the exposed portion of the first and second connection interfaces are further configured to displace from the first vessel and the second vessel and be replaced by the internally concealed portion of the first and second connection interfaces while a hermetically sealed connection is maintained between the first vessel and the second vessel.

2. The system of claim 1, wherein at least one of said first connection interface and said second connection interface is a film, a plastic, a metal alloy, and combinations thereof.

3. The system of claim 1, wherein at least the exposed portion of the first and/or second connection interface displaces externally.

4. The system of claim 1, wherein at least the exposed portion of the first and/or second connection interface displaces internally within a vessel.

5. The system of claim 1, wherein the internal portion is a first end, a second end or both ends of the connection interface.

6. The system of claim 5, wherein following the engagement between the vessels, at least the exposed portions of the interfaces are displaced from the engagement and replaced by the concealed portions, allowing a sterile connection between the vessels.

7. The system of claim 1, wherein at least one of the first and second vessels include a fluid compartment and a fluid-free compartment.

8. The system of claim 7, wherein the connection interface further comprises an internal portion concealed within the fluid-free compartment of the first and/or the second vessel.

9. The system of claim 8, wherein the internal portion is a first end, a second end or both ends of the connection interface.

10. The system of claim 1, further comprising a pressing member on said first vessel and/or said second vessel configured to press a connection interface of the first vessel and/or second vessel against a wall of the first and/or second vessel, thereby allowing a hermetically sealed aperture and a displaceable connection interface.

11. The system of claim 8, wherein following the engagement between the vessels, at least the exposed portions of at least one of the connection interfaces displaces internally within the fluid-free compartment.

12. The system of claim 1, wherein at least one of the first and second vessels include(s) a rotating mechanism, wherein the connection interface connected to the rotating mechanism and the rotating mechanism configured to displace the connection interface.

13. The system of claim 1, wherein at least one of the first and second vessels include(s) a rotating mechanism configured to rotate within the vessel, wherein the rotating mechanism includes a conduit, the conduit allows the fluid passageway upon said rotation.

14. The system of claim 13, wherein the conduit includes a valve for controlling the fluid passageway between the vessels.

15. The system of claim 12, wherein the rotating mechanism includes a handle extending externally from an opening of the vessel and configured to be rotated by a user such to allow the displacement of the first connection interface and/or second connection interface.

16. The system of claim 13, wherein the rotating mechanism includes a handle extending externally from an opening of the vessel and configured to be rotated by a user such to allow the displacement of the first connection interface and/or second connection interface.

17. The system of claim 1, further comprising a piercing member on said first vessel and/or said second vessel configured to pierce a sealed aperture of said first vessel and/or said second vessel, thereby allowing fluid passageway between the vessels.

18. The system of claim 1, wherein an audible sound is emitted when said first vessel engages with said second vessel and/or when said first interface and said second interface are displaced.

19. The system of claim 1, wherein, upon the engagement between said first vessel and said second vessel, the exposed portions of the first connection interface and the second connection interface are configured to displace with respect to the first vessel and the second vessel, thereby removing contaminants between the vessels.

20. The system of claim 1, wherein the decontamination system results in air particle-free engagement of said first vessel and said second vessel, wherein air particles interfacing the interfaces are entrapped by the exposed portions of the interfaces.

21. The system of claim 1, further comprising an engagement mechanism configured to provide a secure engagement between the vessels.

22. The system of claim 21, wherein the engagement mechanism is located on the first vessel and/or on the second vessel.

23. The system of claim 21, wherein the engagement mechanism is selected from a thread, a luer, an adhesive, a clamping arm, a hook and a dent, and a ratchet teeth mechanism.

24. The system of claim 21, wherein when said first vessel and said second vessel engage via the engagement mechanism, a sealed aperture is formed between the vessels.

25. The system of claim 24, wherein the sealed aperture is configured to allow for the displacement of at least one of the first and second connection interface through the sealed aperture while maintaining the hermetically sealed connection between the first vessel and the second vessel.

26. The system of claim 1, further comprising a rail mechanism on at least one of the first and second vessels, the rail mechanism configured to provide the displacement of the connection interface.

27. The system of claim 26, wherein said first connection interface and/or said second connection interface are externally displaced via a sliding motion.

28. The system of claim 1, wherein said first connection interface and said second connection interface displace at about the same time.

29. The system of claim 3, further comprising a tab on at least one of said first connection interface and said second connection interface, the tab configured to allow for an external displacement of said first connection interface and said second connection interface.

30. The system of claim 29, wherein said tab is configured to be pulled axially by a user, the axial pulling of the tab configured to displace said first connection interface and said second connection interface from said first vessel and said second vessel while maintaining the hermetically sealed connection between said first vessel and said second vessel.

31. The system of claim 1, wherein pulling of at least one of the first and second connection interfaces externally displaces both the first and second connection interfaces.

32. The system of claim 1, wherein at least one of the first and second vessel is a container.

33. The system of claim 1, wherein the first vessel is a bag and the second vessel is a vial.

34. The system of claim 1, wherein the first vessel is a bottle and the second vessel is a vial.

35. The system of claim 1, wherein the first vessel is a container and the second vessel is a syringe.

36. A system for decontaminating a surface of vessels, the system comprising:
a first connection interface attached to a first vessel; and
a second connection interface attached to a second vessel,
wherein the first connection interface and the second connection interface having a portion that is externally exposed,
wherein said first vessel and said second vessel are configured to engage with each other, and
wherein the externally exposed portion of the first and second connection interface are further configured to displace from the first vessel and the second vessel into an internal compartment of at least one of the first and second vessels while a hermetically sealed connection is maintained between the first vessel and the second vessel.

37. The system of claim 36, wherein the first vessel is a container and the second vessel is a container.

38. The system of claim 36, wherein the first vessel is a bag and the second vessel is a vial.

39. The system of claim 36, wherein the first vessel is a container and the second vessel is a syringe.

40. A vessel having a connection interface, the connection interface characterized in that a portion of the connection interface is concealed within the vessel and a portion of the connection interface is externally exposed, the externally exposed portion of the connection interface configured to be displaced and replaced by the concealed portion of the connection interface.

41. The vessel of claim 40, wherein the vessel is a container.
